SEQUENCE LISTING (10 sequences)

SEQ ID NO: 1 - DNA, 1997 bp, Arabidopsis thaliana
agcctagttttggaaacatgtggacctctaggctctaagttttcatttgttgtggattctggcatttggctgttttctaacacacaactctggtgtaacggtaccattatcccagtttaaatatgtataatgcatgtttatatatatatatataaaaattaaagactaatgataagaagaaatcaatcaataattgattttttaagtactagatttttttctttcagtttttgacatcctagatttccagaaaaacttgaaaaactatagctaacatgaacgaatatagcaaacatttttttttgtaggaacgaattagtaaacatgaatactctgtaattattgagaagttaattatggattcgtctgggcaatacccattgatatttcccttgaaacaaagatatttgtaataagaacataagtactggacagacacacagagctgaactagacatgtaaattatttacaaatgttttaaataagtgaaaacgttgatttgaaaaatatattagaagattcatttaaacctacatctaggataagccttgagcaatatgtttcatatatatagtataaattgtcatactgatagaaaattgcatatgtgtgtgttaggctttgtaaattagtttcaagtatgaatcaatatctatgatgaagtaagcacattcgtacgaagacgtaaaatggatatatcacgttcgaaagcagcaaccaaagccaaaagtggtcacatggtttgttgttgtaaaatatatatttttggttataattattgtagaataattgagactagaacttaattagtattacgattccgctaattacgcagcccttatctttcgagactataactttaatattacgattcctctgatagtctaattatgcatgcatttttgtgtgtttgcgttttagtttatgttttatacgtaaggtaatatctattcacttacaaatgaagcgataagcttataattttgtagcttgtacccgtagacacaataaaaatgtatatttatcggtgtaatgttttactatgttcggcaacattttaacaacactttagttttcagtgattatataacagctagaaatgaacatatatatagtgttgatcattgaaataaaaacagctttctggtctgagatgtataaaacaaaaataatggatattttatcccgaaagattatgtagagattggatgaacaaattactggttgaacttgaaattattggactttttttttttaaatgtttttgggcagctatacgagaattccatgggtctttgtccacaaaatgttggcgaccatacgaaaacatcgtcgacattcgacaaagagaatctagtcttcataactaaaatatttttaatccaaaaacaaaaataacatccgacgtacgtgaagtaaaaccagtgtttttcttccatcaaattttgcaactatcataagtttttatataaaaacgtgtgtttatccaaacattgagttgatacggccgctaaattttgatattgcaaatgcatgcatgcgaagaattctcacaacgagtagtaaattaatcaaatattaacacgaaaggtactttataataactattactaaaccgtactaatattttgataaacatgaatcgataaattcttgtctaatcatctgccaaagaaacccaatattagagcgcagcctaattaaaatatttgtgttttgtttttttaccccagctgccattgatgaaccacacgatcaaaccgttgacgaggggtttataatctaatcaaacggtgtaaaaccggtgaaacacgcgtgtacatacaccatcccgcatttgataattttagattttaagtttaaaaaatcctgaggatgttgccttgaaagagtctctcattgacttgactctttcctataaatacacctttccactctctcttctccactgtcaaatcaaatccctcttcctcttcaattctctgtttcctccaaa

SEQ ID NO: 2 - DNA, 1134 bp, Arabidopsis thaliana
atggccgtaagtttcgtaacaacatctcctgaggaagaagacaaaccgaagctaggccttggaaatattcaaactccgttaatcttcaacccttcaatgcttaaccttcaagccaatatcccaaaccaattcatctggcctgacgacgaaaaaccttccatcaacgttctcgagcttgatgttcctctcatcgaccttcaaaaccttctctctgatccatcctccactttagatgcttcgagactgatctctgaggcctgtaagaagcacggtttcttcctcgtggtcaatcacggcatcagcgaggagcttatttcagacgctcatgaatacacgagccgcttctttgatatgcctctctccgaaaaacagagggttcttagaaaatccggtgagagtgttggctacgcaagcagtttcaccggacgcttctccaccaagcttccatggaaggagaccctttctttccggttttgcgacgacatgagccgctcaaaatccgttcaagattacttctgcgatgcgttgggacatgggtttcagccatttgggaaggtgtatcaagagtattgtgaagcaatgagttctctatcactgaagatcatggagcttctggggctaagtttaggcgtaaaacgggactactttagagagtttttcgaagaaaacgattcaataatgagactgaattactaccctccatgtataaaaccagatctcacactaggaacaggacctcattgtgatccaacatctcttaccatccttcaccaagaccatgttaatggccttcaagtctttgtggaaaatcaatggcgctccattcgtcccaaccccaaggcctttgtggtcaatatcggcgatactttcatggctctatcgaacgatagatacaagagctgcttgcaccgggcggtggtgaacagcgagagcgagaggaaatcacttgcattcttcttgtgtccgaaaaaagacagagtagtgacgccaccgagagagcttttggacagcatcacatcaagaagataccctgacttcacatggtctatgttccttgagttcactcagaaacattatagagcagacatgaacactctccaagccttttcagattggctcaccaaacccatctaa

SEQ ID NO: 3 - DNA, 1999 bp, Zea mays
cctatccggctacccgtgtcacgtcccatgagaacccgtgactggcgcccctggcctacgcgttatacaaagccgagactgcctgccggtgccggcctgtgcgccgcaccgtcgtcccaattaatcgtgggctagcagcacagcagaagcacccatccatcagggtcaaggctctccggctcccggcccctgtcacttccgctgcagaaggaatacaacgtgacgattgacgaacgaagggagctcgatagatgagacgtaacggagaagatcctcatccagcgttcatgtcctatcttctatgtactgcattgcttcgttcataacagaacgtcgtccgtgcatgcacagagaactatcgtagcgtctattcaatcccgcacgcgcgtacatgtatgcatgctttctagcatatcggatgtggtgtactcgtgtaccgatgtgcgtacaacccgggaaaaaaaccttatcttccacgagcaaaaaatcaaacttgccattgccaaactctccatgtaccagaccagcgttatcagaaccttgtggctggatatatctctatcgatcgtgtgacacacactgagcagatgcagacatgcagtacagcttttatgttatgtgtttatttgccagcgtttgccgcgtctcatcctacgtatgtatctttctgtcacagttacgactacacgagttagtataacgtgcgcccagtcggccatccatcaacaccggcaggcagcagcagacccatcgatcgccgtccgccttaactgagagtctagctcaagctacaggtgccgctgctgttcacggtggagccgctcatcccaaggtcagaaggggaggaggggccggcagctcgatcgggccgacttgccgagacagcgccagcgccagcgcagctacctgcctacctcggtggtttagtcgcaggaccaggccaggtccgctggatctgcggcgtccagcggcggagcctgacgtcaaacgaaagggccgggggcggcaaaacgcaatggacgtgatcgataatggacgaactgatgcactcttacaacgaaagggccgggggcggcaaaacatgatctatagttacaagtaactacacatgtgagctagcactcttacagcggtagcgaccaacctcgaatgaagaaacgcccaaaacaaaagttatagttttaaaaatgttattcatcttcgtagtcgatagccttttcattttaaacccatctattcaactaaataaatcatgtttgatctctcacactgtttgaaatttgatttttttttcaaactatctcgaataaaaaaccaactaaaataaaagttgtatatcttaaaaaattatataactttataggcagatattttatttgaaataatccatccaagaaaaaaatgattgaattttctcacatctaaaattcaaattcttcaaacgaccacgatggaaaaataaccaaaaggaaactatagatctcataaagtaaaaaaaactttattttaaaacaaaataaacatgaaatggttaagagttaagacgaatatcctgaatggacataattgtcttatgattgtagtggttagaggacataagtgtctttatgagtgtcataaaaatattaactaggtaagtcatttttttttgtttttaacggcacaggtatttcattgagtgttccacgacaaaggtactcaagtgttaagtcgtttaccgaatggcacttttgtacgtataggcataaatcgtttacctgtttgtacagcgatgtcgtgatagataaccctcactccatttttagctggttcgttacacgaatttgttaggtaattgccgtattcctcacagcgggcccatgcgtctcgtcaataaattatatggagctcgtactaatcgtcctcttccggtctgctcttattgctaaacctggaaacctgaagaacaaaaaaaagttctcccctcacgcctatataagtacgccggcccagctcgacc

SEQ ID NO: 4 - DNA, 1161 bp, Zea mays
atggacgccagcccgaccccaccgctccccctccgcgccccaactcccagcattgacctccccgctggcaaggacagggccgacgcggcggctaacaaggccgcggctgtgttcgacctgcgccgggagcccaagatcccggagccattcctgtggccgcacgaagaggcgcggccgacctcggccgcggagctggaggtgccggtggtggacgtgggcgtgctgcgcaatggcgacggcgcggggctccgccgcgccgcggcgcaagtggcggcggcgtgcgcgacgcacgggttcttccaggtgtgcgggcacggcgtggacgcggcgctggggcgcgccgcgctggacggcgccagcgacttcttccggctgccgctggctgagaagcagcgggcccggcgcgtccccggcaccgtgtccgggtacacgagcgcgcacgccgaccggttcgcgtccaagctcccctggaaggagaccctgtccttcggcttccacgacggcgccgcggcgcccgtcgtcgtggactacttcaccggcaccctcggccaagatttcgagccagtggggcgggtgtaccagaggtactgcgaggagatgaaggagctgtcgctgacgatcatggagctgctggagctgagcctgggcgtggagcgcggctactaccgggagttcttcgaggacagccgctccatcatgcggtgcaactactacccgccgtgcccggtgccggagcgcacgctgggcacgggcccgcactgcgaccccacggcgctgaccatcctcctgcaggacgacgtcggcgggctggaggtcctggtggacggcgagtggcgccccgtccggcccgtcccaggcgccatggtcatcaacatcggcgacaccttcatggcgctgtccaacgggcggtacaagagctgcctgcaccgcgcggtggtgaaccggcggcaggagcggcaatcgctggccttcttcctgtgcccgcgcgaggaccgggtggtgcgcccgccggccagcgccgcgccgcggcagtacccggacttcacctgggccgacctcatgcgcttcacgcagcgccactaccgcgccgacacccgcacgctggacgccttcacccgctggctctcccacggcccggcggcggcggctccctgcacctaa

SEQ ID NO: 5 - DNA, 2777 bp, Arabidopsis thaliana
aaaaaagtctttgaatttaaaatatttattttatccttccttctcttcccaattcccatacactatatatataagagctcgtcactcttctttcagattcaaaacctcctgatttcattctttactactaataatcattttttgaaaaaaataaaaaagggacatggttttgccatcttcaacaccgttacaaaccaccggaaagaagacgatatcctcgccggaatataacttcccggtgatcgatttctccttaaacgatcggtcaaaactatcggagaagatcgtaaaagcctgtgaggttaatgggtttttcaaggtgataaaccatggagttaaaccggagataataaagagattcgagcatgaaggagaagagttctttaataaaccggaatcagataagctacgagccggtccggcgagtccgttcggttacggatgcaagaacatcgggttcaacggcgatttgggtgagcttgagtatcttcttctccacgcaaatccgacagctgtcgctgataaatctgaaactatatctcatgatgatcctttcaagttcaggtacgtatacatgtattactatttttttcgtacatattacaaatctttttagcatatgttttgtgtgtatatttattgattcttgaacctactttaatttcttgaatcatattataatagccataaaagaaatatattttaactttatgtcaaaaatttgagtaaaacaaaattaaacataaaggcttaagctaatgtatcaaccaaacaaatccatagtatgagttttggttaacaaattcatataaaccataagctcaatgaagcatgagaaaactgacaaccatttatatgtgtgtgtgtgtatgtgtggaaatggcaatcgttggttcttggttgcgtctacatattattattttttaaaaatatatattctatccatttctgtgtgtgtcaaattgaaagttgacccaactgaaattgatgtggattgtgtgtgacaatgtgacttgtgagaaacctaaaaagagagggagagattgaaagtgagatgcaatgtcgaataatggataatgaccagaagacacggcttttaattattatcagtcttgttcctttattgtcattaaagttgctcatattgttttttccttttgttttaaaattagtacaaattataatcataccaacttaatgccggttcacctgatagtttgttgtgatcaataatctatactataatagttggattttgttattggttcatagtagtatattggtatgtggttacaataacgatggcaaccatttttttctctagataatactaaaatatcaaaattgtgaaagttgcttttgtaaccttgactgcgacatggttgataaatcaaattctctttttaaaaaatccctaatttggtggcatataggttacgtataataaatatattttatctaattcttggtggaaatcgaactataaaatatcattcatgggctccatagtagttataattatttgaaaaattcagattaaacacacacatacgttacaaactaacataataattgaacagacttggtcaattagtggatactgtgcatgaacacagacaatactttgagacgtcgtcaaagaatcaaaattggagacgtcaacgagtaaagatttgacttgttattcctcttcgtcatttttaaaatgacaagttgtaaataaaaagcaggaatcatggggtcctcactttccgaacattcctctgtctcatgtcttttctttttctgttattggtatttatcgtaataataataataatttgaaaatctttcagaaaaaataagaacaacaacccgtctctctgtcaattgttgttgagtgtgttaataaattccacgtgttgtttctcttttattagctcggcgacgaatgattacatacgcacggttagagatttggcatgtgagatcattgatctgacgatcgagaatttatggggacagaagtctagcgaagtgagtgagctgatcagagacgttcgtagcgattcgatcctacggttgaatcactatccaccagcaccgtacgcgttaagtggcgttggtcaaataggtttcggagaacattctgaccctcagatcttgacggtgttaagatccaacgacgtagatggacttgagatttgctcacgtgacggcttgtggatcccaatcccatctgaccctacatgcttcttcgtattggtcggtgactgccttcaggtactaaccacattttgtttcgtaaagacttacaaaatagttttcatcaattaattaattaatgaaataatctttgaaaataggcattgacgaatgggagattcactagcgtgaggcatagggttttagcaaacacagcaaagaagccacgtatgtcggcgatgtactttgcagctccaccgttagaggcgaaaatatcaccgttgccaaaaatggtgtcgccggaaaatccaagacgttataattcattcacatggggtgactacaaaaaagctacatactctctccgtttagatgtccctcgtctcgagtttttcaagactttatagattatattcataccattgaaagctagattttttattttttttttgcttgttgaaaaagaaaatatgattgttggattgttacaaatatataccatgtgaaatgtatagatttagatgaacaaaaagaaaaagaaaattgagttttggtcatttactttgtaatttactaatttcacatgtgaatca

SEQ ID NO: 6 - protein, 329 aa, Arabidopsis thaliana [sequence omitted]

SEQ ID NO: 7 - DNA, 5180 bp, Zea mays
gctcccagcccactatacagtatatacacacatcatcccactccttatcgctatatatagcagtgttgtgtgtccatctctctgctacattgcaccgcatcgtctaagctcgcctgccctttacagcagcttactactacaaagacgacaacacagcgcgcgcgcgccaagctgagctcagctcgtcacgacacgccagcatggtggtgccttccacgacgccggtggtgcgccaagaaacgccaccgccgtcccacgacggcataggcataccgacggtggacctgtcggcgccgggcggccgcggcgcgctgtcgcggcaggtggcgcgcgcgtgcgcgcagcacggcttcttccgggccgtgaaccacggcgtggcgccaggccccgccgcacggctggacgccgccgccaggacgttcttcgcgctggcgccgcacaacaagcagcgcgccggcccgcccagcccactcggatacggctgccgcagcatcggcttcaacggcgacgccggggagctcgagtacctgctgctccacgccaacccggccgccgtcgcgcacagggccagatccatcgacactgatgacccctcgcgcttcaggtataacaaatacacggctactatacgtactctcactactctcgtatgcagtacactaccacttgactatatatgtatccatgcatgtgtacatctttgtgcaaaaatcacacatgtatttaaattgatgcatgctgtacttctagctaataagattttagcgacaagcatatatatacacccgtgaaatgattgctggctagttaatgcgaaaccaataaaccatgcacattgtgttgttctattgcattgtacctaaagttttcatcagtattatgttgttattccaacttcgtaataaaaaacaagtatgctactgttggggtgtgtttggcacgggattccagcttcaacatggagctacggtgtgtataatgtaaatttaaacggttttgaaaaaataatctaaatacaaaataaaatgacttatttaaatatctctaaaagtttgcaacttcaacttcacgattttctgaagcttttaatggcatgcttcatcaacttcaccaaatttgtgaagctgaaacagtcccaaacaagacctatatatctgtcgtcgcacactcgaacggtcttggtaaattatgaggtttaattaaacggtattttaaagtggtgcctttatttgtgttggtttgttcatttttaagcttagccgtgttttttttttgctttgttttgtctttccgttgctgaaaagttgaaggacgaaactaatcaaaattcatgactaaaggtaaaaatgaggaaatagagattttggcgaaaggaaccaataaaggttttgtgaggtgtgttagtgttgatggctacctgcaggtgtcaagctcctcggcactgtgcctgcagtgctccttaattaaccaacagcctatgctgatgatagtgtgatagatgatgtggtttgcatatcatagtcctacacctgtcggaatgtctcccgcttgatgctaagctctcagcagctagcagatgagtaggtttatttttgcaaggttctcctagcttttgtgtgtatggaaggtgatgcaagcatttgatatatactaaccttttgctgaatattatagttaaggacttaaggtcgacatgatgtgtacatgcatgcatgtttggtatagttttgttaattcgtccttctcattttaaaagagcacttggggtgtcaatgtgtcatgtatgcactcctctggaatttccaactgaggctgctccccttcttgttgcttatctctaattttggctgtcctgtaccaatttctgatatatatccacataatgttaaaaaggattgcaaaaaatgtagattttgtgaggtgttccttgaatttttttagtatatacagatttaattaagtccaacatgaagcatatagtgttggactttctaagaaggcatcctttgataattgatagtgcgattttttccctgacagaaaagagagtaaagttcgacagcaatgtggccatttgaaatccaaacacatattaatctgaacagtataatggataaaaaaagatagtttattcaagattcttactcataatttttcaactttactagtttttctagtaattaatatgctagcttttatccatgactatgatagctttagggggatgttcctgtacagttcgtggtagaattagtaccctttgctgtcatagtcaggaaggccccccatcaaataaattgcgcaaatgcataactacatggttttcaagacaataattatcgcaataatggcacaattgatgccacaatatgcagggcaatgatatgttagttccaacaattcaagagcatacatggtagtagctagtttctaaagcaaaagaaaaggcacaaactgatgggacataagaaacttgacagctccgcagaatggtcttggacgattttctgatgaaagtttggtataattgccttgccaaaagaacattcgaaaggcttgcaatcggctgctatcattttgcattgaaaaatggcttaaacattttgcttgtgatagggaagcccttgtccatgcatgctgggaaaaacattgaaatgcctaacagttcttttttttgtgaccgagtaaattcttaataacaattgactgaacataacagatgagatatctttaacacaccacacaggccacaggcatgcagtggcctaattcaatagagttggctgagagcaaataaaatgaaagggacaagaaaaccctcctctctcgctctaaggtagttagaagaccatctcggtgtcaggtccctctccaacaaactatatatgtgtattttgcatgtcaatcagtctcacatatctgcggtgcagtggtcttcaattaatggccgtacccaaccaccaattgatgcggctagcaacagaacccatggctattggatagctcttacagctttttttcagccaagtacactaagttccatacagaaaacgatgtttttagatggccacacaggatgtacatcacatacatgaacttcgaaatgaactccctttgttctctgtcaactagtcatgaatcattaatatattgccagtgttccaatactcataatgtagcaccaaactaattaagaagtaggaggttgttcattgccttgatcacattaatataattgtaaaatgcttggcaacaagaacaaaaacagaaatggcatcagaatctttgcagtagccattttgccatggaacacacacacacacacaggtataggtaacagcaagtagggtccacagaagtacaaaagcttgataggaaataaaatgctccccagggccccctaaagagcatatccatgctatccatataaagccaagcaattgaaaaggaggcacacatgcaacgggtcctctttatggcaagatgaatgagatgtcccggaggagggaggtgggagtggcagcaggggcagtgatgagtgattccaaacactgatgggtctatccatggagcagtggaaagagcatgcccaatcctgtcgagagaaacccatttgcccgctggaaaatcggtgacaccaagctatccatgcatgcatcacaagaacctatagcatgtgactggattctgcttctgctgcatagcccagcagatgcatcaagcagctaccgtcttttcaatgctgcagcttttgagttatgctgcatattcatatatacgtcaatcagtcaaaaccttctcatctttcttctgtctcctttttttctctgtcatgggctcatggcacacatccaactggtcttcgtgataaatccattcttttgatccaattggtcttctcacatgctgatgcttttttttttttccttcactgttgctgcagcaatgtggtgaacgagtatgtgggagcgatgaggcagctcgcatgtgagatcctggacctgctcggagaggggctagggctcaaggaccccagatccttcagcaggctcatcgctgacacagacagcgactcactcctgcggataaaccactaccctccaccatgcgccattcacaagcttgaccacgacagtcagtgcaggatgaagaacagtttcaggatcgtggcaggcaatggcgcgaaccagtcggcaggtgcacggatcggattcggggagcactcagacccgcagatccttagcttgctccgatcaaacgatgttgacggccttcaggtgcttctgaacagcgacggcagagaggtgtgggttcaggtgccagccgatccatcagcgttctttgtcaatgttggtgatctcctccaggtaaaataaacaggcactgcggctagtagattaggtaaatgagaattgcttctatatatgagctaccaacgactgtgtcttctaaacagacaaccaggttaattagcaattgctcctttttttgtgaaaagatgggtcagcactgatgttttttggacagcatatgcaccaagtacatcgtaccaactgacaaggatttctgtttttgtcttgttagttgttaccacatctgaatctaacaacattaatccattccatggtgcaacaatataatgtaatatcatgtctcttttcctttttcttgtatgtatgttcccaggccttcacaaatgggaaggtgataagcgtccggcacagggtaattgcaagctcgagcagggcgaggctgtcaacgatctacttcgctgcgccgccactgcatgcacgaattttggccctcccagagacagtcacagccaactcacccaggcagtaccggcccttcacctgggccgagtacaagaagacaatgtactccctccgcctcagccacagccgcctcaacctcttccacatcggccatgatgaccacggcaaaggagaacaagaatagggagaaagtggctgaactgcaggcagagagtttatgttggaggcttaggtaggtagaattgtccccaaaattttggctctctggcctccacagccagataatgtctagcagtatgctggttctgccaagtgccaatgggtaatagtatttagcattgtacacaagtccttgttgtatggaactgatgtactattgcctgttttagcagtttgttgtctcca

SEQ ID NO: 8 - protein, 366 aa, Zea mays
MVVPSTTPVVRQETPPPSHDGIGIPTVDLSAPGGRGALSRQVARACAQHGFFRAVNHGVAPGPAARLDAAARTFFALAPHNKQRAGPPSPLGYGCRSIGFNGDAGELEYLLLHANPAAVAHRARSIDTDDPSRFSNVVNEYVGAMRQLACEILDLLGEGLGLKDPRSFSRLIADTDSDSLLRINHYPPPCAIHKLDHDSQCRMKNSFRIVAGNGANQSAGARIGFGEHSDPQILSLLRSNDVDGLQVLLNSDGREVWVQVPADPSAFFVNVGDLLQAFTNGKVISVRHRVIASSSRARLSTIYFAAPPLHARILALPETVTANSPRQYRPFTWAEYKKTMYSLRLSHSRLNLFHIGHDDHGKGEQE

SEQ ID NO: 9 - DNA, 3893 bp, Zea mays
atggtggtgccttccacgacgccggtggtgcgccagcaaacgacgccgccgcagtcatcccacgccggcggcataccgacggtggacctatcggcgcacggcggccgcggcgctctgtcgcggcaggtggtgcgcgcgtgcgcggagcacggcttcttccgggccgtgaaccacggcgtgccgccgggccccgccgcgcggctggacgccgccgccaggacgttcttcgcgctggcgccgcgcgacaagcagcgcgccggcccgcctagcccgctcggctacggctgccgtagcatcggcttcaacggcgacgccggggagctcgagtacctgctgctccacgccaacaacccggccgccgtcgcgcacagggccagggccatcgacgcggaggagccctcgcgattcaggtataacaaacaatacacggctacgtagtctcactacgctcgtatatatcatgcatgtacattgtgtagagttgtttttatttagctgtcgtattgcattgtcgttaagttttgatcaacattgttcttccagctttatttagagcatcttcaagactctttattttttactctctatttgactatttatctctacataaatattaaactatatatataatatcttattccaacaacatataagttagcttggatagcgaaagttgctagtcaaatttagctagtgagagagtcaatttagagagctggataggttgacaaattaaatggctagtttgttggagagctattttgatataaaaatagctaaaatttagcttgacaaactatttagctagtctcttggagatgccttggagatgccagctggttttatgagccatggtaatctatgctaggccttttgaggtttaaaaatttttataagaaaggcatacgccaaaggaacgaacagttgttatgcaggtgcagtgtgttaatattgatggctacctggagagtcatgctcctctgcactgtgcctgcagtgctctttaattaaccaacagcctatatatgctgatgacactagtgtgatcgatagatgtggtttgcgtatcatagtcctacacctgtcggaatatctcgcttttgacgagtgatgcaagcattatttgggatagaaaagcgaacagtatatatggttcggcaccaatataatgggggcactcaatcaaaatacaagcacacattaatttgaatgtcacactgtaacggataacagaaaagaaagatagttcactcaagaaagataattcttacccataacaatttcactgtactgatcttttcttttagttcaatgcatttatatatccgtgaccgtgatagctctagcgaatgtatctttagagtttgggtagagctagtatgccatgttgtcatagtcagaaaggcccccatcaaataaattatgcaaatgcatgactgcatagtttcctaatcaatgatcaataatgcaactattgatgccacaataggcagagcaatgatatgttagttccaacaattcaagagcatacacatggatgtagccagtttctaagcaaaaaaagaaaaaggcacaaactgatcgatggggcataacagacttgacagctccacagaatgaatgggcttggtcaattttccgacgaaagttgggcacaattgccttgccacaagaacattcgaaagcacacccggcttacaatcgcctgcctaacaactgagtgggcataacagatgagatatctttagctgtttctgtgcatttggaaggagaaagctaagatcccatccaaacgacactgctatgctacgagagatctcacacgctacacacaggccaggcacaggcagtggcctagttcaatgctagagtagttggttgagggcaaataaaatgaaagggacaagaaaaccctcccagcctctatggtagttcaaagacccctctcagtgtcaggtcgtccctctccagcagactatgtattaatatatgtgtgtatttgcatgccaatcagtctcacacagctgcgggcggtgcagtagtcttcaattaatggccatacgcccatacccaaccgccaattgatgcagatgctagcaacagaaagggcccatggcttatttagtgaagtactctaagtaccatgcagaaaatgatgtgttgatacattcgtacatgaactctgaaacgaattccctttgtcaactagacatgaatcattaatacattgtcaatgttgtaatatttatagtatatcagctattaagaagtagaaggttgttagctgtcttgaccacatagaaaaaaaatctaagcttacagttaagaatgatagcaacaaagagtaaaaacaggaatgacatcagaatctatgccgtagccatttttccatggagcacatgtgtacgtaatacgttatccgttttttttatttgtcatgttttagttaaaaaataaattagtcggcgacaaatatttgagaacagggtagtaccaaataggatccatacaagtacaaaagcttgacaggaaataagattgtgtttggtttgagaattcagtccatccaaaataaggtgatacatcatgagtctatttctcaaatttgatgtaacgagtgcaaaaagactaaaccaatccatccgtggagaagtgcaaaaggcacgcccaatcctgtctaaaaacacccccatttgcccattggaaaactggtcacaagaaccgaatatgtacagcatgtgacttgtgttctggttctgctgcgtagcctggatgcatccagagcatcgagctatccgtgttttaacggctgcagcttttgagtcactgcatatccatacatcaaacaagtcaaattttttcatctttcttcagtcttctttttctcttgtcatggcacacatccaattggtcttcacgacagctccattctcttgatccaattgatcttgggcagaatttaactcacatgttggtgccttttcttccctgttgttgcagtaatgtggtgaacgagtacgtgggagcaatgaggcagcttgcatgtgagatcctggacctgttaggagaggggctagggctcgaggaccccaggtccttcagcaagctcatcaccgacacagacagcgactcactcctgaggatcaaccactaccctacagcatgcaacgctcacaaccttgaccatgacagccagtgcaagatgaagagcagcgtcagaaccaagactactagcaatggcgtgaagccgtcggcaggtggacgggtcggattcggggagcattcagacccgcagattcttagcttgcttcgagcaaatgacgtcgacggccttcaggtgcttctgaacgccgacggcaaggaggtgtgggttcaggtgccggccgatcagtcagcgttctttgtcaatgttggtgacctcctccaggtaaaggcattgatttacttgtaaataaatcagtactagtagattaactgaatgaaattgcttcttggagcagcttagaaattaaattagcagactctgaatccaacaataaccaaacactaatacatggcgatattatctctcatacttggcttgaattcatctcttttccctttcttggtcgcaggctttaacaaatgggaagctggtaagtgtccggcacagggtaattgcaagctccagcagggcaaggctgtcgacaatctacttcgcagcgccgccactgcatgcgcgaattttggccctcgcagagacgatcacagccaacgcacccagccagtaccggcccttcacctgggccgagtacaagaagacaatgtactcgctccgcctgagccacagccgcctcaacctcttccacatcgaccatgatggccacagcaatgtcggtgaaggagaagaatag

SEQ ID NO: 10 - protein, 371 aa, Zea mays
MVVPSTTPVVRQQTTPPQSSHAGGIPTVDLSAHGGRGALSRQVVRACAEHGFFRAVNHGVPPGPAARLDAAARTFFALAPRDKQRAGPPSPLGYGCRSIGFNGDAGELEYLLLHANNPAAVAHRARAIDAEEPSRFSNVVNEYVGAMRQLACEILDLLGEGLGLEDPRSFSKLITDTDSDSLLRINHYPTACNAHNLDHDSQCKMKSSVRTKTTSNGVKPSAGGRVGFGEHSDPQILSLLRANDVDGLQVLLNADGKEVWVQVPADQSAFFVNVGDLLQALTNGKLVSVRHRVIASSSRARLSTIYFAAPPLHARILALAETITANAPSQYRPFTWAEYKKTMYSLRLSHSRLNLFHIDHDGHSNVGEGEE

(19) United States
(12) Patent Application Publication
(10) Pub. No.: US 2014/0344996 A1
(43) Pub. Date: Nov. 20, 2014

(54) METHODS AND MEANS TO PRODUCE ABIOTIC STRESS TOLERANT PLANTS

(71) Applicants: Dirk Gustaaf Inze, Moorsel-Aalst (BE); Marieke Dubois, Bouschepe (FR); Aleksandra Skirycz, Belem (BR)

(72) Inventors: Dirk Gustaaf Inze, Moorsel-Aalst (BE); Marieke Dubois, Bouschepe (FR); Aleksandra Skirycz, Belem (BR)

(73) Assignee: VIB VZW, Gent (BE)

(21) Appl. No.: 14/345,227

(22) PCT Filed: Sep. 14, 2012
(86) PCT No.: PCT/EP2012/068100

§ 371 Date: Jul. 9, 2014

Related U.S. Application Data
(60) Provisional application No. 61/627,033, filed on Sep. 16, 2011.

(30) Foreign Application Priority Data
Sep. 19, 2011 (GB) 1116129.6

(51) Int. Cl. C12N 15/82

(52) U.S. Cl. CPC C12N 15/8271; C12N 15/8216
USPC 800/278; 536/23.6; 800/298; 800/320.1; 800/320.2; 800/320.3; 800/320; 435/419; 435/412; 536/23.2

(57) ABSTRACT

This disclosure relates to the field of plant molecular biology and concerns methods for enhancing the abiotic stress tolerance in plants by modulating the expression of a gene involved in the gibberellin biosynthesis during the period of abiotic stress. This disclosure also provides chimeric constructs useful in the methods disclosed herein. In addition, transgenic plants having an enhanced abiotic stress resistance are provided herein.

### CROSS-REFERENCE TO RELATED APPLICATIONS

This application is a national phase entry under 35 U.S.C. §371 of International Patent Application PCT/EP2012/068100, filed Sep. 14, 2012, designating the United States of America and published in English as International Patent Publication WO 2013/037959 A1 on Mar. 21, 2013, which claims the benefit under Article 8 of the Patent Cooperation Treaty and under 35 U.S.C. §119(e) to U.S. Provisional Patent Application Ser. No. 61/627,033, filed Sep. 16, 2011, and to Great Britain Patent Application Serial No. 1116129.6, filed Sep. 19, 2011, the disclosure of each of which is hereby incorporated herein in its entirety by this reference.

### TECHNICAL FIELD

This disclosure relates to the field of plant molecular biology, more particularly to the field of agriculture, and concerns methods for enhancing the abiotic stress tolerance in plants by modulating the gibberellin biosynthesis during the period of abiotic stress. This disclosure also provides chimeric constructs useful in the methods described herein. In addition, transgenic plants having an enhanced abiotic stress resistance are provided herein.

### BACKGROUND

Abiotic stress is defined as the negative impact of non-living factors on the living organisms in a specific environment. The non-living variable must influence the environment beyond its normal range of variation to adversely affect the population performance or individual physiology of the organism in a significant way. Abiotic stress is essentially unavoidable. Abiotic stress affects animals, but plants are especially dependent on environmental factors, so it is particularly constraining. Abiotic stress is the most harmful factor concerning the growth and productivity of crops worldwide. Drought, temperature extremes, and saline soils are the most common abiotic stresses that plants encounter. Globally, approximately 22% of agricultural land is saline and areas under drought are already expanding and this is expected to increase further. Other crops are exposed to multiple stresses, and the manner in which a plant senses and responds to different environmental factors appears to be overlapping.

The most obvious detriment concerning abiotic stress involves farming. It has been calculated that abiotic stress causes the most crop loss of any other factor and that most major crops are reduced in their yield by more than 50% from their potential yield. In addition, it has been speculated that this yield reduction will only worsen with the dramatic climate changes expected in the future. Because abiotic stress is widely considered a detrimental effect, the research on this branch of the issue is extensive. When subjected to environmental stress, plants actively reduce their vegetative growth to save and redistribute resources and thus increase their chance of survival when the stress becomes severe. However, when the stress does not threaten survival, growth inhibition can be seen as counter-productive because it leads to an unnecessary drop in productivity and substantial yield penalties. “Bolder” plants that are able to grow during mild stress episodes might prove an efficient way to boost productivity in regions that do not experience severe weather conditions. Therefore, understanding the mechanisms underlying growth inhibition in response to stress can lead to entry points for interfering with the stress-induced growth reductions.

In plants, organ growth is driven by two tightly controlled and dynamic processes: cell proliferation and subsequent cell expansion. The coordination of these two processes during leaf growth ultimately determines leaf size and shape. In dicots, such as the model species Arabidopsis thaliana, leaves initiate at the flank of the meristem and, in the initial phase, their growth is driven exclusively by cell proliferation. In somewhat older leaves, cells will exit the mitotic cell cycle and start expanding from the tip onward. This transition is manifested by the onset of endoreduplication, which is a modified cell cycle in which replication proceeds without mitosis with higher ploidy levels as a consequence. In water-limited environments, plants respond by a rapid initial growth reduction followed by growth adaptation, resulting in leaves with fewer and smaller cells.

Transcriptional changes upon drought are already well understood and extensively described in mature plant organs. It is, however, known in the art that these transcriptional changes are extremely different in mature vs. growing plant organs. Exclusively in fully proliferating leaves, the induction of the Ethylene Response Factor 6 (ERF6) was already observed 1.5 hours after exposure to in vitro osmotic stress. In particular, it was found that ERF6 regulates the transcription of a number of signaling genes. One of these targets is an up-regulated enzyme responsible for gibberellin inactivation in plants. It was hypothesized that the transient decrease in gibberellic acid (GA) during the period of abiotic stress was responsible for the inhibition of cell expansion and division and, hence, it was surprisingly shown that the expression of a chimeric construct directing GA biosynthesis expression under control of a stress-regulated promoter was sufficient to overcome the growth inhibition under abiotic stress. Thus, the disclosure described herein provides dedicated chimeric genes that, upon transformation in plants, lead to an up-regulation of the gibberellic acid synthesis only during the period of abiotic stress.

### DISCLOSURE

To facilitate the understanding of this disclosure, a number of terms are defined below. Terms defined herein (unless otherwise specified) have meanings as commonly understood by a person of ordinary skill in the areas relevant to the disclosure described herein. As used in this specification and its appended claims, terms such as “a,” “an,” and “the” are not intended to refer to only a singular entity, but include the general class of which a specific example may be used for illustration, unless the context dictates otherwise. The terminology herein is used to describe specific embodiments, but their usage does not delimit this disclosure, except as outlined in the claims.

Despite its importance for agriculture, environmental stress-induced growth inhibition, which is responsible for significant yield reductions, is only poorly understood. In the present disclosure, the molecular mechanisms underlying cell cycle inhibition in young proliferating leaves of the model plant Arabidopsis thaliana were unraveled when subjected to mild osmotic stress. A detailed cellular analysis demonstrated that as soon as osmotic stress is sensed, cell cycle progression rapidly arrests, but cells are kept in an ambivalent state allowing a quick recovery (“pause”). Remarkably, it was found that cell cycle arrest coincides with an increase in the Ethylene Response Factor 6 (ERF6). Careful study showed that one of the target genes of ERF6 is GA2ox6, an enzyme responsible for gibberellin degradation in plants.

Thus, in the present disclosure, it is shown that the degradation of gibberellins production (or stimulating gibberellins catabolism) in plants is a prime signal responsible for growth arrest during stress exposure and thus substantially contributes to stress-associated growth penalty and yield losses. It was shown that transiently up-regulating the gibberellins levels (during the period perceived as abiotic stress) can, therefore, relieve observed growth repression and thus limit yield losses. Since gibberellins have pleiotropic effects on plant growth and development, and ectopic modification of gibberellins may result in a number of undesirable phenotypes, there was a need to transiently stimulate the gibberellin production in plants, i.e., only during the period of abiotic stress. This was achieved by constructing dedicated chimeric genes for up-regulation of specific genes in the gibberellins biosynthesis. Surprisingly, it was shown that temporarily inducing gibberellin production during the period of abiotic stress encountered by the plant had the effect of overcoming the growth arrest imposed by the abiotic stress.

Accordingly, this disclosure provides for a method for producing an abiotic stress-tolerant plant relative to a control plant, by increasing the production of gibberellins in the plant during the period of abiotic stress imposed on the plants comprising introducing and expressing in the plant a chimeric gene comprising an abiotic stress-inducible promoter operably linked to a gibberellin biosynthesis gene. In a particular embodiment, the abiotic stress (or environmental stress, which is equivalent) is mild stress. The meaning of “mild stress” is apparent from the text of the application and the further appended examples.

A “gibberellin biosynthesis gene” is a gene that encodes a gene product that is involved in the synthesis (e.g., in the plant cell) of gibberellins. All known gibberellins are diterpenoid acids that are synthesized by the terpenoid pathway in plastids and then modified in the endoplasmic reticulum and cytosol until they reach their biologically active form. All gibberellins are derived via the ent-gibberellane skeleton, but are synthesized via ent-kaurene. The gibberellins are named GA1, GA2, . . . , GAₙ in order of discovery. For example, Gibberellic acid, which was the first gibberellin to be structurally characterized, is GA3. Examples of gibberellin biosynthesis genes that can be used to carry out the disclosure described herein comprise “GA 20 oxidase 1,” “GA 3 oxidase 1 (GA 3ox1)” and “ent kaurenoic acid oxidase.”

GA 3ox1 or gibberellin 3-oxidase 1 catalyzes the later steps in the synthesis of bioactive gibberellins. A representative member of GA 3ox1 is derived from Arabidopsis thaliana with the AGI-code At1g15550. Other representative members (orthologous genes) are derived from rice (database accession number OS01G08220), Sorghum (SB03G004020), corn (ZM03G02800) and soybean (GM13G43850 and GM15G01500).

GA 20ox1 or gibberellin 20-oxidase 1 is considered as one of the rate-limiting steps in the synthesis of bioactive gibberellins. A representative member of GA 20ox1 is derived from Arabidopsis thaliana with the AGI-code At4g25420. Other representative members (orthologous genes) are derived from rice (database accession number OS03G63970), soybean (database accession numbers GM16G32550, GM20G29210 and GM09G27490) and corn (database accession number ZM03G20130).

Ent-kaurenoic acid oxidase is encoded by two redundant genes in Arabidopsis thaliana, KAO1 (At1g05160) and KAO2 (At2g32440). Other orthologous genes are from corn (ZM09G19030), Sorghum (SB10G000920), soybean (GM15G14330 and GM09G03400) and rice (OS06G02019).

“Abiotic stress-inducible promoters” that can be used in the context of the disclosure described herein are promoters that are derived from genes that are up-regulated under abiotic stress. Preferably, abiotic stress-inducible promoters are derived from genes that are up-regulated between 1 and 5 hours after the plant experiences abiotic stress. The person skilled in the art can readily identify abiotic stress-inducible genes (i.e., genes that are induced upon the induction of abiotic stress) by various methods described in the art, such as, for example, microarray analysis. Promoters can be identified from abiotic stress-inducible genes by isolating, for example, 500 to 3000 base pairs, preferably 1000 to 2000 base pairs upstream of the start-codon of abiotic stress-inducible genes. A person skilled in the art disposes of several tools to test the functionality of promoters (and, hence, if the isolated fragment of an abiotic stress-inducible promoter is sufficient). Such tools for studying promoter functionality are further described herein. Preferred examples of promoters that can be used in the context of the present disclosure are promoters derived from the list of genes consisting of ERF6 (representative member is At4g17490), TCH3 (representative member is At2g41100), embryo-abundant protein-related (representative member is At1g55450), ankyrin repeat family protein (representative member is At2g24600), gene with unknown function (representative member is At1g05575), calcium-binding protein (representative member is At2g46600), glycine-rich protein (At5g28630), gene with unknown function (representative member is At1g19020), EDA39 (representative member is At4g33050), STZ (representative member is At1g27730), MYB51 (representative member is At1g18570), WRKY33 (representative member is At2g38470), ERF1 (representative member is At4g17500), ERF2 (representative member is At5g47220, ERF5 (representative member is At5g47230), and ERF11 (representative member is At1g28370).

Preferred promoters are promoters derived from STZ, MYB51, ERF6 and WRKY33.

A particularly preferred promoter is derived from the ERF6 gene.

It is understood that the promoter can also be a promoter derived from an orthologous gene. For example, the promoter derived from the ERF6 gene can be the 2000 base pairs sequence upstream of the start-codon of the ERF6 open reading frame, which is derived from the At4g17490 gene, but the promoter can also be derived from the Zea mays orthologous gene ZM05G29170 (see further outlined in Examples 6 and 7). Methods for isolating promoters from orthologous genes are well known to the person skilled in the art.

In the art, it is shown that particular gibberellin biosynthesis genes can function in different plants, such as a heterologous gene (i.e., a gene derived from one plant species or genus can be used to function in a different plant species or genus), can be used in the chimeric gene construct (e.g., At4g25420 can be used to express and to function in the gibberellin synthesis pathway in corn). In some instances, it might be useful to adapt the codon usage when a gene from one plant (e.g., a dicotyledonous plant) is used to express in another plant (e.g., a monocotyledonous plant). In other instances, it might be preferred to isolate the plant orthologous gene (e.g., the corn homologue of At1g15550 can be used) for expression in corn as outlined in Example 7. Methods for isolation of orthologous genes from other genomes or gene libraries are well known to the person skilled in the art.

In yet another embodiment, a chimeric gene is provided comprising the following operably linked DNA elements: a) an abiotic stress-inducible promoter, b) a DNA region encoding for a gibberellin biosynthesis gene, and c) a 3′ end region comprising transcription termination and polyadenylation signals functioning in cells of the plant.

In yet another embodiment, a chimeric gene is provided comprising the following operably linked DNA elements: a) an abiotic stress-inducible promoter selected from the list of genes consisting of ERF6, TCH3, embryo-abundant protein-related, ankyrin repeat family protein, At1g05575, calcium-binding protein, glycine-rich protein, At1g19020, EDA39, STZ, MYB51, WRKY33, ERF1, ERF2, ERF5 and ERF11, or an orthologous gene thereof, b) a DNA region encoding for a gibberellin biosynthesis gene selected from the list consisting of GA20ox1, ent kaurenoic acid oxidase and GA3ox and c) a 3′ end region comprising transcription termination and polyadenylation signals functioning in cells of the plant.

In yet another embodiment, a transgenic plant or a transgenic seed or a transgenic plant cell is provided comprising a chimeric gene as described before.

Particularly preferred transgenic plants, seeds or plant cells disclosed herein comprise a crop plant or a monocot or a cereal such as rice, wheat, maize, barley, millet, rye, triticale, sorghum emmer, spelt, secale, einkorn and oats.

A “chimeric gene” or “chimeric construct” is a recombinant nucleic acid sequence in which a promoter or regulatory nucleic acid sequence is operatively linked to, or associated with, a nucleic acid sequence that codes for an mRNA, such that the regulatory nucleic acid sequence is able to regulate transcription or expression of the associated nucleic acid coding sequence. The regulatory nucleic acid sequence of the chimeric gene is not normally operatively linked to the associated nucleic acid sequence as found in nature.

In the present disclosure, a “plant promoter” comprises regulatory elements, which mediate the expression of a coding sequence segment in plant cells. For expression in plants, the nucleic acid molecule must be linked operably to or comprise a suitable promoter that expresses the gene at the right point in time and with the required spatial expression pattern. In a preferred aspect, the plant promoter of the disclosure is induced when the plant encounters the abiotic stress. A preferred promoter is an abiotic stress-inducible promoter. Also preferred is a promoter that is an abiotic stress-inducible promoter that is active during the cell proliferation phase of the plant.

For the identification of functionally equivalent promoters, the promoter strength and/or expression pattern of a candidate promoter may be analyzed, for example, by operably linking the promoter to a reporter gene and assaying the expression level and pattern of the reporter gene in various tissues of the plant. Suitable well-known reporter genes include, for example, beta-glucuronidase or beta-galactosidase. The promoter activity is assayed by measuring the enzymatic activity of the beta-glucuronidase or beta-galactosidase. The promoter strength and/or expression pattern may then be compared to that of a reference promoter (such as the one used in the methods disclosed herein). Alternatively, promoter strength may be assayed by quantifying mRNA levels or by comparing mRNA levels of the nucleic acid used in the methods disclosed herein, with mRNA levels of housekeeping genes such as 18S rRNA, using methods known in the art, such as Northern blotting with densitometric analysis of autoradiograms, quantitative real-time PCR or RT-PCR (Heid et al., 1996 Genome Methods 6:986-994). Generally, by “weak promoter” is intended a promoter that drives expression of a coding sequence at a low level. By “low level” is intended at levels of about 1/10,000 transcripts to about 1/100,000 transcripts, to about 1/500,000 transcripts per cell. Conversely, a “strong promoter” drives expression of a coding sequence at high level, or at about 1/10 transcripts to about 1/100 transcripts to about 1/1000 transcripts per cell. Generally, by “medium strength promoter” is intended a promoter that drives expression of a coding sequence at a lower level than a strong promoter, in particular, at a level that is in all instances below that obtained when under the control of a 35S CaMV promoter.

The term “operably linked” as used herein refers to a functional linkage between the promoter sequence and the gene of interest, such that the promoter sequence is able to initiate transcription of the gene of interest.

A “constitutive promoter” refers to a promoter that is transcriptionally active during most, but not necessarily all, phases of growth and development and under most environmental conditions, in at least one cell, tissue or organ. In the present disclosure, a constitutive promoter is not preferred because of the pleiotropic effects of ethylene on plant growth.

The term “terminator” encompasses a control sequence that is a DNA sequence at the end of a transcriptional unit, which signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or, alternatively, from another plant gene, or less preferably from any other eukaryotic gene.

In yet another aspect, the down-regulation of the GA2ox6 gene is envisaged, wherein an inhibitory RNA molecule directed against the GA2ox6 nucleotide sequence is expressed in a plant or plant cell or plant seed under the control of a stress-inducible promoter.

In yet another embodiment, a chimeric gene is provided comprising the following elements:
i) A plant-expressible promoter that is a stress-inducible promoter,
ii) A DNA region that, when transcribed, yields a GA2ox6-inhibitory RNA molecule;
iii) A 3′ end region involved in transcription termination and polyadenylation.

In a specific embodiment, the stress-inducible promoter is derived from the genes selected from the list consisting of ERF6, TCH3, embryo-abundant protein-related, ankyrin repeat family protein, At1g05575, calcium-binding protein, glycine-rich protein, At1g19020, EDA39, STZ, MYB51, WRKY33n ERF1, ERF2, ERF5 and ERF11 or orthologous genes thereof.

In yet another specific embodiment, the DNA region present in the chimeric gene comprises a nucleotide sequence selected from the groups consisting of:
a. A nucleotide sequence of at least 19 out of 20 consecutive nucleotides from a nucleotide sequence encoding a protein comprising the amino acid sequence of SEQ ID NOS:6, 8, or 10;
b. A nucleotide sequence of at least 19 out of 20 consecutive nucleotides from the complement of a nucleotide sequence encoding a protein comprising the amino acid sequence of SEQ ID NOS:6, 8 or 10:
c. A nucleotide sequence of at least 19 out of 20 consecutive nucleotides of a nucleotide sequence of SEQ ID NOS:5, 7 or 9; and
d. A nucleotide sequence of at least 19 out of 20 consecutive nucleotides of the complement of a nucleotide sequence of SEQ ID NOS:5, 7 or 9.

In yet another embodiment, a plant or plant cell or seed or propagating material is provided comprising the above-described chimeric gene for down-regulating the GA2ox6 gene.

It will be appreciated for the person skilled in the art that many methods are known in the art for the generation (or production) of an inhibitory RNA molecule directed against the GA2ox6 nucleotide sequence. Non-limiting examples of methods that can be used for reducing the expression of the GA2ox6 gene comprise co-suppression, antisense suppression, hairpin RNA interference, ribozyme expression directed against GA2ox6, or artificial microRNA directed against GA2ox6 nucleotide sequence. SEQ ID NO:5 depicts the Arabidopsis thaliana genomic sequence of GA2ox6, SEQ ID NO:7 depicts the genomic sequence of a Zea mays ortholog of the Arabidopsis GA2ox6. SEQ ID NO:9 depicts the genomic sequence of a second Zea mays ortholog of the Arabidopsis GA2ox6.

“Selectable marker,” “selectable marker gene,” or “reporter gene” includes any gene that confers a phenotype on a cell in which it is expressed to facilitate the identification and/or selection of cells that are transfected or transformed with a nucleic acid construct disclosed herein. These marker genes enable the identification of a successful transfer of the nucleic acid molecules via a series of different principles. Suitable markers may be selected from markers that confer antibiotic or herbicide resistance, that introduce a new metabolic trait, or that allow visual selection. Examples of selectable marker genes include genes conferring resistance to antibiotics (such as nptII that phosphorylates neomycin and kanamycin, or hpt, phosphorylating hygromycin, or genes conferring resistance to, for example, bleomycin, streptomycin, tetracyclin, chloramphenicol, ampicillin, gentamycin, geneticin (G418), spectinomycin or blasticidin), to herbicides (for example, bar, which provides resistance to BASTA®; aroA or gox providing resistance against glyphosate, or the genes conferring resistance to, for example, imidazolinone, phosphinothricin or sulfonylurea), or genes that provide a metabolic trait (such as manA that allows plants to use mannose as sole carbon source or xylose isomerase for the utilization of xylose, or anti-nutritive markers such as the resistance to 2-deoxyglucose). Expression of visual marker genes results in the formation of color (for example, β-glucuronidase, GUS or β-galactosidase with its colored substrates, for example, X-Gal), luminescence (such as the luciferin/luciferase system) or fluorescence (Green Fluorescent Protein, GFP, and derivatives thereof). This list represents only a small number of possible markers. The skilled worker is familiar with such markers. Different markers are preferred, depending on the organism and the selection method.

It is known that upon stable or transient integration of nucleic acids into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells, together with the gene of interest. These markers can, for example, be used in mutants in which these genes are not functional by, for example, deletion by conventional methods. Furthermore, nucleic acid molecules encoding a selectable marker can be introduced into a host cell on the same vector that comprises the sequence encoding the polypeptides of this disclosure or used in the methods disclosed herein, or else in a separate vector. Cells that have been stably transfected with the introduced nucleic acid can be identified, for example, by selection (for example, cells that have integrated the selectable marker survive, whereas the other cells die).

Since the marker genes, particularly genes for resistance to antibiotics and herbicides, are no longer required or are undesired in the transgenic host cell once the nucleic acids have been introduced successfully, the process according to this disclosure for introducing the nucleic acids advantageously employs techniques that enable the removal or excision of these marker genes. One such method is what is known as “co-transformation.” The co-transformation method employs two vectors simultaneously for the transformation, one vector bearing the nucleic acid according to this disclosure and a second bearing the marker gene(s). A large proportion of transformants receives or, in the case of plants, comprises (up to 40% or more of the transformants), both vectors. In case of transformation with Agrobacteria, the transformants usually receive only a part of the vector, i.e., the sequence flanked by the T-DNA, which usually represents the expression cassette. The marker genes can subsequently be removed from the transformed plant by performing crosses.

In another method, marker genes integrated into a transposon are used for the transformation together with desired nucleic acid (known as the Ac/Ds technology). The transformants can be crossed with a transposase source or the transformants are transformed with a nucleic acid construct conferring expression of a transposase, transiently or stable. In some cases (approximately 10%), the transposon jumps out of the genome of the host cell once transformation has taken place successfully and is lost. In a further number of cases, the transposon jumps to a different location. In these cases, the marker gene must be eliminated by performing crosses. In microbiology, techniques were developed that make possible, or facilitate, the detection of such events. A further advantageous method relies on what is known as recombination systems; whose advantage is that elimination by crossing can be dispensed with. The best-known system of this type is what is known as the Cre/lox system. Crel is a recombinase that removes the sequences located between the loxP sequences. If the marker gene is integrated between the loxP sequences, it is removed once transformation has taken place successfully, by expression of the recombinase. Further recombination systems are the HIN/HIX, FLP/FRT and REP/STB system (Tribble et al., J. Biol. Chem. 275, 2000: 22255-22267; Velmurugan et al., J. Cell. Biol. 149, 2000: 553-566). A site-specific integration into the plant genome of the nucleic acid sequences according to this disclosure is possible.

For the purposes of this disclosure, “transgenic,” “transgene,” or “recombinant” means with regard to, for example, a nucleic acid sequence, an expression cassette, gene construct or a vector comprising the nucleic acid sequence or an organism transformed with the nucleic acid sequences, expression cassettes or vectors according to this disclosure.

A “transgenic plant” for the purposes of this disclosure is thus understood as meaning, as above, that the nucleic acids used in the method of this disclosure are not present in, or originating from, the genome of the plant, or are present in the genome of the plant but not at their natural locus in the genome of the plant, it being possible for the nucleic acids to be expressed homologously or heterologously. However, as mentioned, transgenic also means that, while the nucleic acids according to this disclosure or used in the disclosed method are at their natural position in the genome of a plant, the sequence has been modified with regard to the natural sequence, and/or that the regulatory sequences of the natural sequences have been modified. “Transgenic” is preferably understood as meaning the expression of the nucleic acids according to this disclosure at an unnatural locus in the genome, i.e., homologous, or heterologous expression of the nucleic acids takes place. Preferred transgenic plants are mentioned herein.

For the purpose of this disclosure, related or orthologous genes of the gibberellin biosynthesis pathway as described hereinbefore can be isolated from the publicly available sequence databases. In addition, promoters derived from orthologous genes (as described hereinbefore) can be identified in sequence databases. The “sequence identity” of two related nucleotide or amino acid sequences, expressed as a percentage, refers to the number of positions in the two optimally aligned sequences that have identical residues (×100) divided by the number of positions compared. A gap, i.e., a position in an alignment where a residue is present in one sequence but not in the other, is regarded as a position with non-identical residues. The alignment of the two sequences is performed by the Needleman and Wunsch algorithm (Needleman and Wunsch (1970) J. Mol. Biol. 48:443-453). The computer-assisted sequence alignment above can be conveniently performed using a standard software program such as GAP, which is part of the Wisconsin Package Version 10.1 (Genetics Computer Group, Madison, Wis., USA) using the default scoring matrix with a gap creation penalty of 50 and a gap extension penalty of 3. Sequences are indicated as “essentially similar” when such sequence have a sequence identity of at least about 75%, particularly at least about 80%, more particularly at least about 85%, quite particularly about 90%, especially about 95%, more especially about 100%, quite especially are identical. It is clear that when RNA sequences are essentially similar or have a certain degree of sequence identity with DNA sequences, thymine (T) in the DNA sequence is considered equal to uracil (U) in the RNA sequence.

Alternatively, the skilled person can isolate orthologous plant genes involved in the gibberellin biosynthesis or promoters derived from genes activated under abiotic stress through methods of genetic hybridization. Such methods are well known to the skilled (plant) molecular biologist.

The term “modulation” means, in relation to expression or gene expression, a process in which the expression level is changed by the gene expression in comparison to the control plant, the expression level may be increased or decreased. The original, unmodulated expression may be of any kind of expression of a structural RNA (rRNA, tRNA) or mRNA with subsequent translation. For the purposes of this disclosure, the original unmodulated expression may also be absence of any expression. The term “modulating the activity” shall mean any change of the expression of the disclosed nucleic acid sequences or encoded proteins, which leads to increased yield and/or increased growth of the plants. The expression can increase from zero (absence of, or immeasurable expression) to a certain amount, or can decrease from a certain amount to immeasurable small amounts or zero.

The term “expression” or “gene expression” means the transcription of a specific gene or specific genes or specific genetic construct. The term “expression” or “gene expression,” in particular, means the transcription of a gene or genes or genetic construct into structural RNA (rRNA, tRNA) or mRNA with or without subsequent translation of the latter into a protein. The process includes transcription of DNA and processing of the resulting mRNA product.

The term “increased expression” or “overexpression” as used herein means any form of expression that is additional to the original wild-type expression level. For the purposes of this disclosure, the original wild-type expression level might also be zero, i.e., absence of expression or immeasurable expression.

Methods for increasing expression of genes or gene products are well documented in the art and include, for example, overexpression driven by appropriate promoters (as described hereinbefore), the use of transcription enhancers or translation enhancers. Isolated nucleic acids that serve as promoter or enhancer elements may be introduced in an appropriate position (typically upstream) of a non-heterologous form of a polynucleotide so as to up-regulate expression of a nucleic acid encoding the polypeptide of interest. If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added may be derived from, for example, the nopaline synthase or octopine synthase genes, or, alternatively, from another plant gene, or less preferably from any other eukaryotic gene.

An intron sequence may also be added to the 5′ untranslated region (UTR) or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg (1988) Mol. Cell. Biol. 8:4395-4405; Callis et al. (1987) Genes Dev. 1:1183-1200). Such intron enhancement of gene expression is typically greatest when placed near the 5′ end of the transcription unit. Use of the maize introns Adh1-S intron 1, 2, and 6, the Bronze-1 intron are known in the art. For general information, see The Maize Handbook, Chapter 1 16, Freeling and Walbot, Eds., Springer, N.Y. (1994).

The term “introduction” or “transformation” as referred to herein encompass the transfer of an exogenous polynucleotide into a host cell, irrespective of the method used for transfer. Plant tissue capable of subsequent clonal propagation, whether by organogenesis or embryogenesis, may be transformed with a genetic construct of the present disclosure and a whole plant regenerated therefrom. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (F. A. Krens et al. (1982), Nature 296:72-74; I. Negrutiu et al. (1987), Plant Mol. Biol. 8:363-373); electroporation of protoplasts (R. D. Shillito et al. (1985), Bio/Technol. 3:1099-1102); microinjection into plant material (A. Crossway et al. (1986), Mol. Gen. Genet. 202:179-185); DNA or RNA-coated particle bombardment (T. M. Klein et al. (1987), Nature 327:70); infection with (non-integrative) viruses and the like.

Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. It has proved particularly expedient in accordance with this disclosure to allow a suspension of transformed agrobacteria to act on the intact plant or at least on the flower primordia. The plant is subsequently grown on until the seeds of the treated plant are obtained (Clough and Bent, Plant J. (1998) 16:735-743). Methods for Agrobacterium-mediated transformation of rice include well-known methods for rice transformation, such as those described in any of the following: European patent application EPI 198985, Aldemita and Hodges (Planta 199:612-617, 1996); Chan et al. (Plant Mol. Biol. 22(3):491-506, 1993), Hiei et al. (Plant J. 6(2):271-282, 1994), which disclosures are incorporated by reference herein as if fully set forth. In the case of corn transformation, the preferred method is as described in either Ishida et al. (Nat. Biotechnol. 14(6):745-50, 1996) or Frame et al. (Plant Physiol. 129(1):13-22, 2002), which disclosures are incorporated by reference herein as if fully set forth. The methods are further described by way of example in B. Jenes et al., “Techniques for Gene Transfer” in: Transgenic Plants, Vol. 1, Engineering and Utilization, eds. S.D. Kung and R. Wu, Academic Press (1993) 128-143; and in Potrykus Annu. Rev. Plant Physiol. Plant Molec. Biol. 42 (1991) 205-225.

The nucleic acids or the construct to be expressed is preferably cloned into a vector, which is suitable for transforming Agrobacterium tumefaciens, for example, pBin19 (Bevan et al. (1984) Nucl. Acids Res. 12-8711). Agrobacteria transformed by such a vector can then be used in a known manner for the transformation of plants, such as plants used as a model, like Arabidopsis (Arabidopsis thaliana is, within the scope of the present disclosure, not considered as a crop plant), or crop plants such as, by way of example, tobacco plants, for example, by immersing bruised leaves or chopped leaves in an agrobacterial solution and then culturing them in suitable media. The transformation of plants by means of Agrobacterium tumefaciens is described, for example, by Hofgen and Willmitzer in Nucl. Acid Res. (1988) 16:9877, or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

In addition to the transformation of somatic cells, which then have to be regenerated into intact plants, it is also possible to transform the cells of plant meristems and, in particular, those cells that develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic (K. A. Feldman and M. D. Marks (1987), gMol. Gen. Genet. 208:1-9; K. Feldmann (1992), In: C. Koncz, N.-H. Chua and J. Shell, eds, Methods in Arabidopsis Research, Word Scientific, Singapore, pp. 274-289).

Alternative methods are based on the repeated removal of the influorescences and incubation of the excision site in the center of the rosette with transformed agrobacteria, whereby transformed seeds can likewise be obtained at a later point in time (Chang (1994), Plant J. 5:551-558; Katavic (1994), Mol. Gen. Genet. 245:363-370). However, an especially effective method is the vacuum infiltration method with its modifications such as the “floral dip” method. In the case of vacuum infiltration of Arabidopsis, intact plants under reduced pressure are treated with an agrobacterial suspension (N. Bechthold (1993), CR Acad. Sci. Paris Life Sci. 316:1194-1199), while in the case of the “floral dip” method the developing floral tissue is incubated briefly with a surfactant-treated agrobacterial suspension (S. J. Clough and A. F. Bent (1998), The Plant J. 16:735-743).

A certain proportion of transgenic seeds are harvested in both cases, and these seeds can be distinguished from non-transgenic seeds by growing under the above-described selective conditions. In addition, the stable transformation of plastids is advantageous because plastids are inherited maternally in most crops, reducing or eliminating the risk of transgene flow through pollen. The transformation of the chloroplast genome is generally achieved by a process that has been schematically displayed in Klaus et al., 2004 (Nature Biotechnology 22(2):225-229). Briefly, the sequences to be transformed are cloned together with a selectable marker gene between flanking sequences homologous to the chloroplast genome. These homologous flanking sequences direct site-specific integration into the plastome.

Plastidal transformation has been described for many different plant species and an overview is given in Bock (2001), “Transgenic plastids in basic research and plant biotechnology,” J. Mol. Biol. 2001 Sep. 21; 312 (3):425-38, or P. Maliga (2003), “Progress towards commercialization of plastid transformation technology,” Trends Biotechnol. 21:20-28. Further biotechnological progress has recently been reported in the form of marker-free plastid transformants, which can be produced by a transient co-integrated maker gene (Klaus et al., 2004, Nature Biotechnology 22(2):225-229).

The genetically modified plant cells can be regenerated via all methods with which the skilled worker is familiar. Suitable methods can be found in the above-mentioned publications by S. D. Kung and R. Wu, Potrykus or Hofgen and Willmitzer.

Generally, after transformation, plant cells or cell groupings are selected for the presence of one or more markers that are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance, using Southern analysis, for the presence of the gene of interest, copy number and/or genomic organization. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); or grafts of transformed and untransformed tissues (e.g., in plants, a transformed rootstock grafted to an untransformed scion).

Usually, an increase in yield and/or growth rate occurs when the plant is under non-stress conditions. Plants typically respond to exposure to stress by growing more slowly. In conditions of severe stress, the plant may even stop growing altogether. Mild stress, on the other hand, is defined herein as being any stress to which a plant is exposed that does result in the plant ceasing to grow slower (or temporarily) but still has the capacity to resume growth when the (mild) stress disappears. Mild stress in the sense of this disclosure leads to a reduction in the growth of the stressed plants of less than 40%, 35%, 30% or 25%, more preferably less than 20% or 15% in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. “Mild stresses” are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures.

“Biotic stresses” are typically those stresses caused by pathogens, such as bacteria, viruses, fungi, nematodes and insects.

The “abiotic stress” may be an osmotic stress caused by a water stress, e.g., due to drought, salt stress, or freezing stress. Abiotic stress may also be an oxidative stress or a cold stress. “Freezing stress” is intended to refer to stress due to freezing temperatures, i.e., temperatures at which available water molecules freeze and turn into ice. “Cold stress,” also called “chilling stress,” is intended to refer to cold temperatures, e.g., temperatures below 10°, or preferably below 5° C., but at which water molecules do not freeze. As reported in Wang et al. (Planta (2003) 218:1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol. (2003), 133:1755-1767) describes a particularly high degree of “cross talk” between drought stress and high-salinity stress. For example, drought and/or salinization are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signaling pathways and cellular responses, such as the production of stress proteins, up-regulation of anti-oxidants, accumulation of compatible solutes and growth arrest.

The term “non-stress” conditions as used herein are those environmental conditions that allow optimal growth of plants. Persons skilled in the art are aware of normal soil conditions and climatic conditions for a given location. Plants with optimal growth conditions (grown under non-stress conditions) typically yield in increasing order of preference at least 97%, 95%, 92%, 90%, 87%, 85%, 83%, 80%, 77% or 75% of the average production of such plant in a given environment. Average production may be calculated on harvest and/or season basis. Persons skilled in the art are aware of average yield productions of a crop.

In particular, the methods of the present disclosure may be performed under non-stress conditions. In an example, the methods of the present disclosure may be performed under non-stress conditions such as mild drought to give plants having increased yield relative to control plants.

In another embodiment, the methods of the present disclosure may be performed under stress conditions.

In an example, the methods of the present disclosure may be performed under stress conditions such as drought to give plants having increased yield relative to control plants. In another example, the methods of the present disclosure may be performed under stress conditions such as nutrient deficiency to give plants having increased yield relative to control plants.

Nutrient deficiency may result from a lack of nutrients such as nitrogen, phosphates and other phosphorous-containing compounds, potassium, calcium, magnesium, manganese, iron and boron, amongst others.

In yet another example, the methods of the present disclosure may be performed under stress conditions such as salt stress to give plants having increased yield relative to control plants. The term salt stress is not restricted to common salt (NaCl), but may be any one or more of: NaCl, KCl, LiCl, MgCl₂, CaCl₂, amongst others.

In yet another example, the methods of the present disclosure may be performed under stress conditions such as cold stress or freezing stress to give plants having increased yield relative to control plants.

The terms “increase,” “improve,” or “enhance” are interchangeable and shall mean in the sense of the application at least a 3%, 4%, 5%, 6%, 7%, 8%, 9% or 10%, preferably at least 15% or 20%, more preferably 25%, 30%, 35% or 40% more yield and/or growth in comparison to control plants as defined herein.

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

Plants that are particularly useful in the methods of this disclosure include, in particular, monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g., Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g., Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g., Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g., Glycine max, Soja hispida or Soja max), Gossypium hirsutum, Helianthus spp. (e.g., Helianthus annuus), Hemerocallis fulva, Hibiscus spp., Hordeum spp. (e.g., Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g., Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g., Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g., Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g., Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

The choice of suitable control plants is a routine part of an experimental setup and may include corresponding wild-type plants or corresponding plants without the gene of interest. The control plant is typically of the same plant species or even of the same variety as the plant to be assessed. The control plant may also be a nullizygote of the plant to be assessed. Nullizygotes are individuals missing the transgene by segregation. A “control plant” as used herein refers not only to whole plants, but also to plant parts, including seeds and seed parts.

The disclosure described herein provides a genetically modified plant, which can be a transgenic plant, that is more tolerant to a stress condition than a corresponding reference plant. As used herein, the term “tolerant” when used in reference to a stress condition of a plant, means that the particular plant, when exposed to a stress condition, shows less of an effect, or no effect, in response to the condition as compared to a corresponding reference plant (naturally occurring wild-type plant or a plant not containing a construct of the present disclosure). As a consequence, a plant encompassed within the present disclosure shows improved agronomic performance as a result of enhanced abiotic stress tolerance and grows better under more widely varying conditions, such as increased biomass and/or higher yields and/or produces more seeds. Preferably, the transgenic plant is capable of substantially normal growth under environmental conditions where the corresponding reference plant shows reduced growth, yield, metabolism or viability, or increased male or female sterility.

As used herein, the term “drought-tolerance” refers to the more desirable productivity of a plant under conditions of water deficit stress. Water deficit stress develops as the evapotranspiration demand for water exceeds the supply of water. Water deficit stress can be of large or small magnitude (e.g., days or weeks of little or no accessible water), but drought tolerant plants will show better growth and/or recovery from the stress, as compared to drought-sensitive plants. As used herein, the term “water use efficiency” refers to the more desirable productivity of a plant per unit of water applied. The applied water may be the result of precipitation or irrigation.

As used herein, the term “salt-tolerance” refers to the more desirable productivity of a plant under conditions of salinity stress. While for each species, the threshold at which soil and/or water salinity (often expressed as conductivity) differs, a salt-tolerant plant would have a higher salinity threshold before yields decline. Salt-tolerance also refers to the sensitivity of yield to water and/or soil salinity beyond the threshold. So a salt-tolerant plant would show less impact on yield per unit of salinity than a salt-sensitive plant. Salt-tolerance refers to an increased threshold and/or a decreased sensitivity beyond the threshold of yield to salinity.

The term “expression cassette” refers to any recombinant expression system for the purpose of expressing a nucleic acid sequence of this disclosure in vitro or in vivo, constitutively or inducibly, in any cell, including, in addition to plant cells, prokaryotic, yeast, fungal, insect or mammalian cells. The term includes linear and circular expression systems. The term includes all vectors. The cassettes can remain episomal or integrate into the host cell genome. The expression cassettes can have the ability to self-replicate or not (i.e., drive only transient expression in a cell). The term includes recombinant expression cassettes that contain only the minimum elements needed for transcription of the recombinant nucleic acid.

The term “inducible” or “inducibly” means the polypeptides of the present disclosure are not expressed, or are expressed at very low levels, in the absence of an inducing agent. The expression of the polypeptides of the present disclosure is greatly induced in response to an inducing agent.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A and 1B: Mannitol response and functional analysis of the putative ERF6 target genes. FIG. 1A: Schematic representation of the induction by mannitol of the putative ERF6 targets. The expression of genes in proliferating leaves at different time points, upon mannitol treatment, was measured by micro-array (A. Skirycz et al. (2010), Plant Physiol. 152:226-244). While the ERF6 expression peaks at 1.5 hours upon stress exposure, most of the ERF6 targets are only induced (FC>2) at later time points. FIG. 1B: BinGo analysis of the 254 putative ERF6 target genes. There is a significant enrichment of genes with roles in both biotic and abiotic stress response, as well as with roles in ethylene-related signaling pathways.

FIGS. 2A and 2B: ERF6 is the direct regulator of MYB51, WRKY33 and STZ. FIG. 2A: Expression analysis of putative target genes upon ERF6 activation in proliferating leaves. Gene expression changes measured by RT-qPCR showed the rapid induction of MYB51, WRKY33 and STZ upon overexpression of ERF6, indicating that they can be primary ERF6 targets. Expression values are normalized to their expression in GFP-IOE line. FIG. 2B: Confirmation of the ERF6 binding to the promoters of MYB51, WRKY33 and STZ by protoplast activation assay. Co-transformation of BY2 protoplast with a 35S-ERF6 vector and respective promoter:luciferase constructs resulted in a two-fold increase of the luciferine signal compared to control. Indicated values are luciferine detection levels normalized to the negative control. **=significantly different from control at 0.001% significance level. For both graphs, error bars indicate standard errors.

FIG. 3: Induction of ERF6 overexpression results in growth inhibition through reduced cell division and expansion. (Panel A) Phenotype of wild-type plant (left), ERF610E-W plant (middle) and ERF610E-S plant (right). Plants overexpressing ERF6 show a severe growth retardation. (Panel B) In soil phenotype of 21-day-old wild-type plant (upper left), ERF610E-W plant (upper right) and ERF610E-S plant (lower row). Scalebar=5 mm. (Panel C) Size of the third leaf of seedlings transferred to Dexamethasone at D9 to induce ERF6 overexpression. Leaf size becomes significantly smaller than control at D11 for ERF6^{IOE}-S and at D12 for ERF610E-W. (Panel D) Left: size of abaxial epidermal cells of harvested third leaves. While cells of control plants significantly expanded between D14 and D16, the cells of the 2 ERF6 overexpressing lines fail to expand sufficiently. Right: average calculated number of cells in the harvested third leaves. (Panel E) Endoreduplication index (EI) of the third leaf subsequently measured upon activation of ERF6 overexpression at 9DAS. The EI represents the average number of endoreduplications performed by each nucleus. In ERF6^{IOE}-S, the EI increases much earlier than in the control line, indicating a faster onset of endoreduplication. Error bars indicate standard errors.

FIG. 4: The growth inhibition by ERF6 occurs through GA signaling. (Panel A) Growth complementation assay. By crossing the two independent ERF6-IOE lines with a 35S-ga20ox1 line (extopic GA overexpression) the dwarf phenotype could be partially or fully complemented in ERF6^{IOE}-S and ERF6^{IOE}-W lines, respectively. (Panel B) Third leaf size measurements of plants described in Panel A. (Panel C) Confocal imaging of root tips of ERF6IOE-S×pRGA:RGA-GFP lines without Dex (left) and with Dex (right). Activation of ERF6 overexpression (right) clearly causes the RGA to be stabilized. (Panel D) Expression level of the transcription factors involved in the stress signaling network in the ERF6-IOE×35S-Ga20-ox1 lines. The expression levels in the crossed lines are as high as in the ERF6-IOE lines.

### DETAILED DESCRIPTION

The following non-limiting Examples describe methods and means according to the disclosure. Unless stated otherwise in the Examples, all techniques are carried out according to protocols standard in the art. The following examples are included to illustrate embodiments of this disclosure. Those of skill in the art should, in light of the present disclosure, appreciate that many changes can be made in the specific embodiments that are disclosed and still obtain a like or similar result without departing from the concept, spirit and scope of this disclosure. More specifically, it will be apparent that certain agents, which are both chemically and physiologically related, may be substituted for the agents described herein while the same or similar results would be achieved. All such similar substitutes and modifications apparent to those skilled in the art are deemed to be within the spirit, scope and concept of the disclosure as defined by the appended claims.

### EXAMPLES

### 1. ERF6 is a Central Regulator of the Osmotic Stress Transcriptional Network

Exposure of Arabidopsis seedlings to low concentration of mannitol induced a massive transcriptional response in developing leaf initials, followed by a rapid growth arrest. The transcription factor ETHYLENE RESPONSE FACTOR 6 was identified as one of the genes that is rapidly up-regulated in proliferating leaf initials of plants exposed to osmotic stress. To investigate which part of the osmotic stress response found in the proliferating leaf primordia is regulated by ERF6, the ERF6 regulon was initially delineated using co-expression networks constructed based on the available micro-array data in CORNET (S. de Bodt et al. (2010), Plant Physiol. 2010 March; 152(3):1167-79. Using a Pearson Correlation cut-off of 0.7, 143 genes were found to be co-expressed with ERF6 in stress datasets. Comparison of the 143 genes obtained by the in silico analysis with the >1500 genes induced upon 1.5, 3, 12, and 24 hours of exposure to low mannitol concentration (A. Skirycz et al. (2010), Plant Physiol. 152:226-244) showed a highly significant overlap of 47 genes (p=7.5*10.26). Among those 47 genes were several well-characterized stress-related genes such as several members of the WRKY, ZCF and MYB families.

To verify the in silico analysis, putative ERF6 target genes were experimentally investigated using glucocorticoid-inducible overexpression (IOE) lines in which overexpression can be activated by a dexamethasone (DEX) treatment. Two independent and homozygous ERF6-IOE lines were selected with a single insertion locus and with different levels of ERF6 overexpression: a strong ERF6 overexpressor (Log 2FC=12.8) called ERF^{IOE}-S and a weaker line (Log 2FC=7.8) called ERF^{IOE}-W. As a control, a GFP-IOE line with similar construct was chosen.

For the target gene analysis, the ERF^{IOE}-S line and the control were grown without inducer until 9DAS, when growth of the third leaf is driven exclusively by cell proliferation, and then transferred to plates with or without dexamethasone. To specifically study the target genes in growing tissue, all subsequent analysis was performed on the micro-dissected third leaf.

To identify the ERF6 target genes, genome-wide microarray analysis was performed on proliferating leaves 4 hours +/−DEX application using the AGRONOMICS tiling arrays. 254 differentially expressed genes (FDR<0.05 after correction for multiple testing) were found. Amongst them, 198 had probes on the ATH I array, which we used previously to study the induction by mannitol. 97 of the 198 tested genes were induced by low mannitol concentrations in actively growing tissues (p<0.05 after correction for multiple testing), which is significantly more than expected by chance (p=2.9*10⁻⁹²).

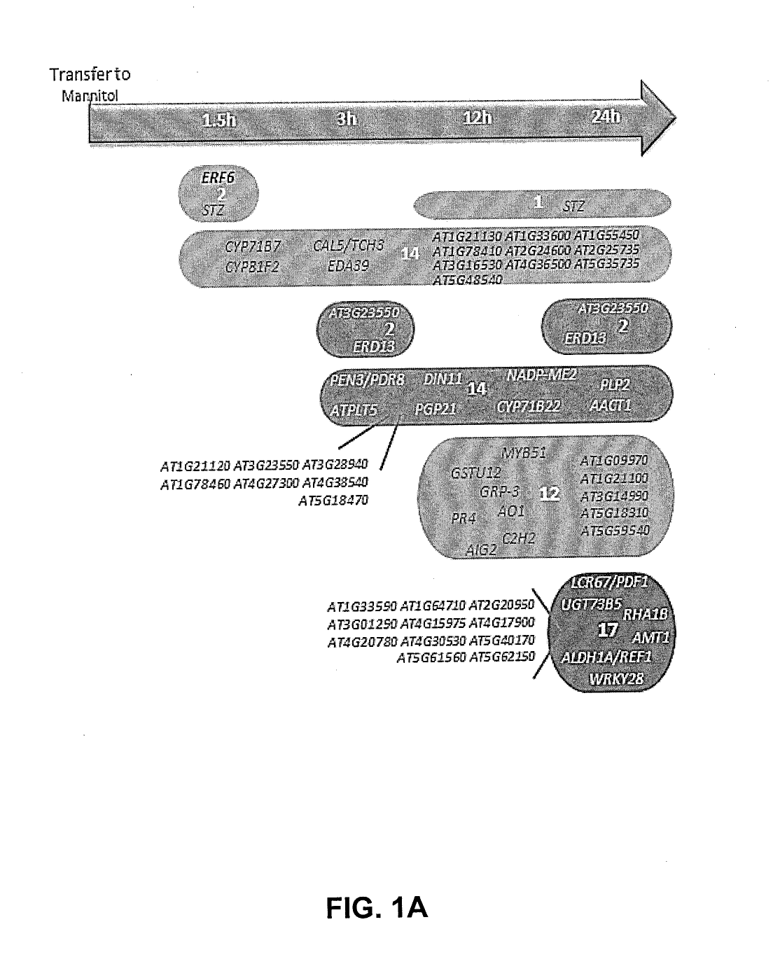

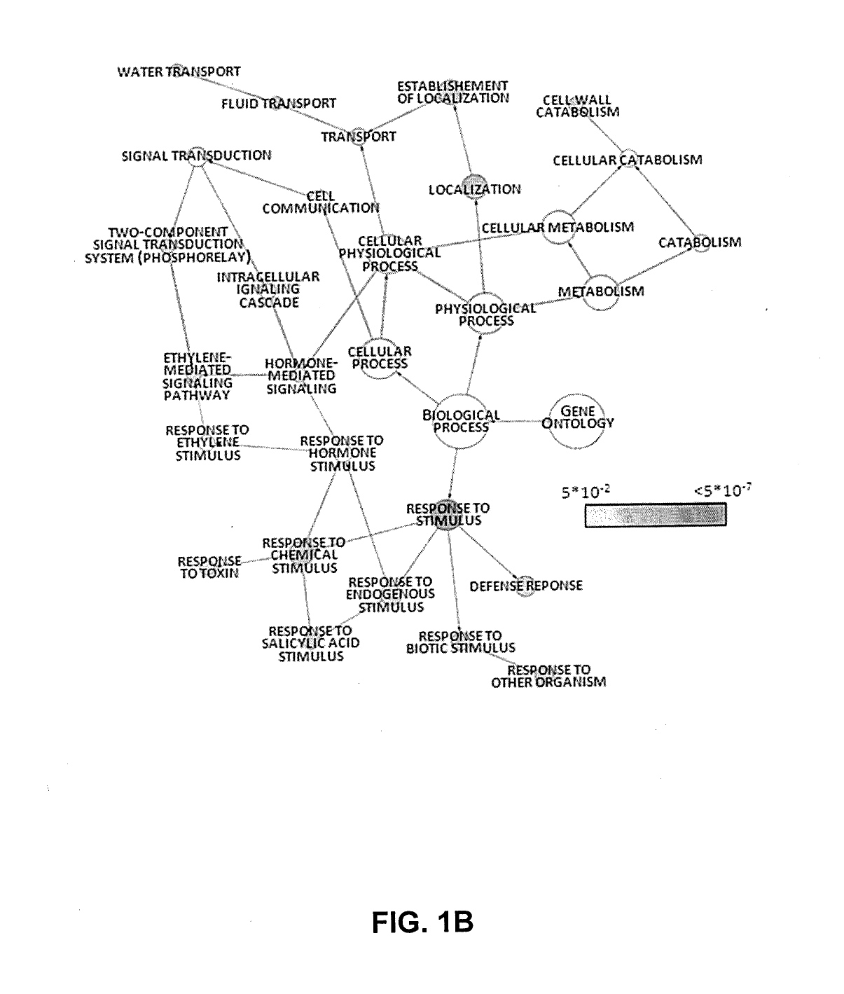

Moreover, analysis of the expression data over time upon mannitol treatment revealed a very fast induction for ERF6 itself (1.5 hours), while the majority of the putative target genes were induced at 3 hours or later (FIG. 1A). GO annotation analysis using BinGo revealed that the putative ERF6 targets are enriched in several stress-related, as well as hormone response-related, functional categories such as: “response to stimulus,” “response to biotic stimulus,” “response to chemical stimulus,” “response to salicylic acid,” “response to ethylene,” “ethylene mediated signaling pathway,” etc. (FIG. 1B).

The putative ERF6 targets are also significantly enriched for direct drought effector genes such as the aquaporins PIP and PIP2. Next to these drought effectors, many of the ERF6 targets are involved in signaling: there are 10 kinases and 22 transcription factors that would further propagate and execute the stress response.

In summary, co-expression analysis followed by experimental work provided a list of putative ERF6 targets, of which the vast majority is also a part of the osmotic stress transcriptional network. As ERF6 expression is induced shortly after stress imposition and as it regulates transcription of many other signaling genes, it can be considered as a central element in the signaling events following stress sensing.

### 2. ERF6 Acts Directly Up-Stream of Stress-Related Transcription Factors

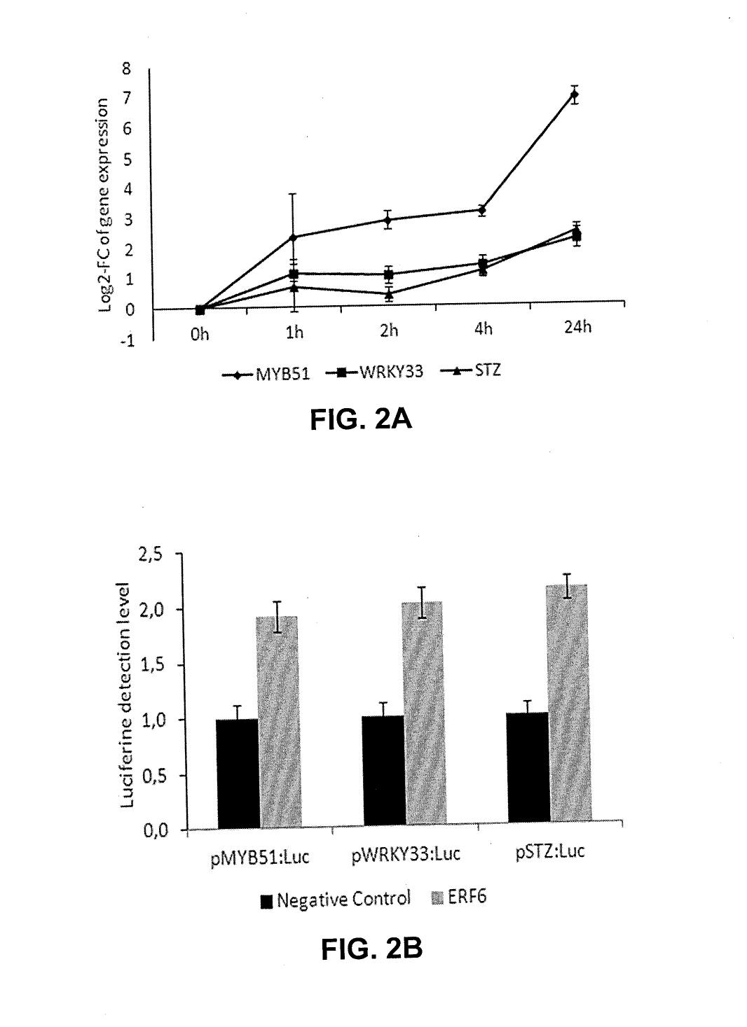

Particularly interesting in the context of osmotic stress regulatory networks but also for the functional characterization of ERF6, were the transcription factors downstream of ERF6. After concentrating on STZ, WRKY33 and MYBS1, they were found to be significantly induced by ERF6. Additional RT-qPCR analysis confirmed the fast induction (within one hour following dexamethasone treatment) of SIZ, WRKY33 and MYB51 upon ERF6 activation (FIG. 2A), suggesting that they are primary ERF6 targets. The ability of ERF6 to induce SIZ, WRKY33 and MYBS1 was confirmed using a protoplast activation assay. For this end, the respective promoters of SIZ, WRKY33 and MYB51 were cloned upstream of the luciferase gene (Luc) and expressed together with a 35S-ERF6 vector in tobacco BY2 protoplasts. Binding of ERF6 to the promoter of interest induces expression of the Luc gene producing luciferine, which is subsequently detected by illuminesence. There was a two-fold increase of the signal for the pSTZ:Luc, pWRKY33:Luc and pMYB51:Luc constructs compared to the negative control, indicating that these transcription factors are strongly and most likely directly induced by ERF6 (FIG. 2B).

### 3. ERF6 Inhibits Cell Proliferation and Expansion

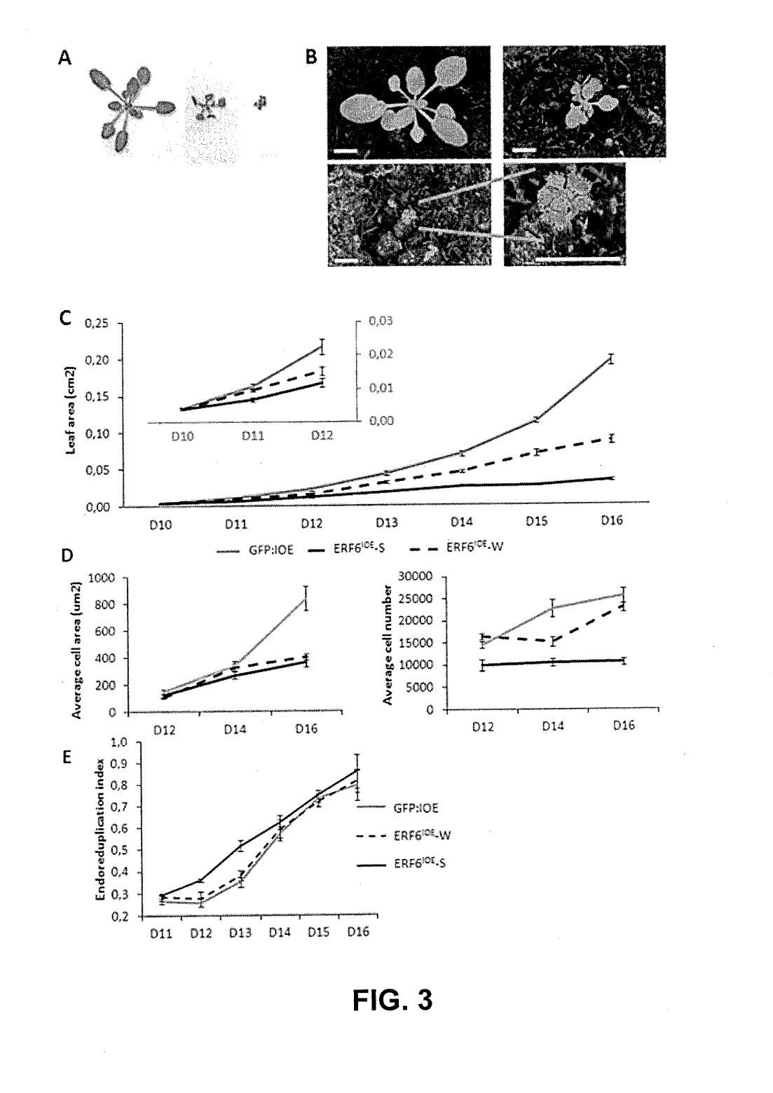

The most striking phenotype of the ERF6:IOE lines when left for growth on DEX in vitro, was a very severe growth retardation (FIG. 3, Panel A). Plants became dwarfed, dark green with epinastic leaves and stunted influorescence. To investigate whether this phenotype would hold in soil as well, Arabidopsis seedlings were sprayed with a 5 μM DEX solution every 2 days. A similar phenotype was observed, although less pronounced (FIG. 3, Panel B). As would be expected, the growth retardation was more severe in the ERF6^{IOE}-S line than in ERF6^{IOE}-W.

To examine this phenotype further, the effect of ERF6 overexpression on the growth of the proliferating third leaf was focused on. Plants were grown on control medium until 9DAS, when the third leaf just initiated and were then transferred to medium with DEX to activate the ERF6 overexpression. Timing of the growth inhibition caused by ERF6 was investigated by harvesting the third leaf daily after transfer to DEX. Leaf areas were measured and cell numbers and sizes calculated from epidermal cell drawings for the selected time points. The first significant reduction of leaf area was measured 48 hours after transfer for ERF6^{IOE}-S, while 72 hours were needed for ERF6^{IOE}-W (FIG. 3, Panel C). At cellular level, a strong decrease in cell area was observed for both ERF6^{IOE}-S and ERF6^{IOE}-W (FIG. 3, Panel D). Furthermore, based on leaf and average cell areas, the number of cells per leaf could be calculated.

A significant decrease in cell number could be observed upon ERF6 overexpression, which was more pronounced in ERF6^{IOE}-S compared to ERF6^{IOE}-W (FIG. 3, Panel D). In conclusion, overexpression of ERF6 limits plant growth by inhibiting both cell division and cell expansion. To further detail the possible underlying cause of the observed growth reduction observed in ERF6:IOE plants, ploidy levels were examined in the third leaf following induction of ERF6 activity by dexamethasone. This revealed a faster onset of endoreduplication in ERF6^{IOE}-S manifested by an earlier increase in 8C and 16C at the expense of 2C nuclei (FIG. 3, Panel F). This suggests that ERF6 activity pushes cells into the differentiation program, resulting in fewer divisions and fewer cells.

Weak overexpression of ERF6 does not affect endoreduplication. Both ERF6:IOE lines also were crossed with the mitotic marker line CYCB1; 1:Dbox-GUS. The obtained seeds were grown on control medium without DEX until 9DAS. At this time point, when the third leaf is emerging from the meristem and thus fully proliferative, half of the seedlings were transferred to DEX to activate ERF6 overexpression. The other seedlings were transferred to control medium without DEX. GUS staining was performed 72 hours after ERF6 activation, at 12DAS. At this time point, the third leaf is in a transitional developmental stage, meaning that the cells at the bottom of the leaf are still proliferating while in the leaf tip, cells stop to divide and enter the cell differentiation phase. In 12DAS third leaves of DEX-treated ERF6^{IOE}-S plants, GUS staining was much weaker indicating a reduced area of proliferating cells (FIG. 3, Panel F). This confirmed that ERF6 causes the cells to exit the cell cycle and shift toward cell differentiation.

In conclusion, the data shows that during early leaf development, ERF6 restrains cell division by stimulating cell cycle exit toward cell expansion. However, in a later developmental stage, ERF6 is able to inhibit cell expansion as well. The combined effects of ERF6 on both cell proliferation and cell expansion reduce leaf size by more than 75% as compared to control leaves.

### 4. ERF6 Affects Growth by Modulating GA Metabolism

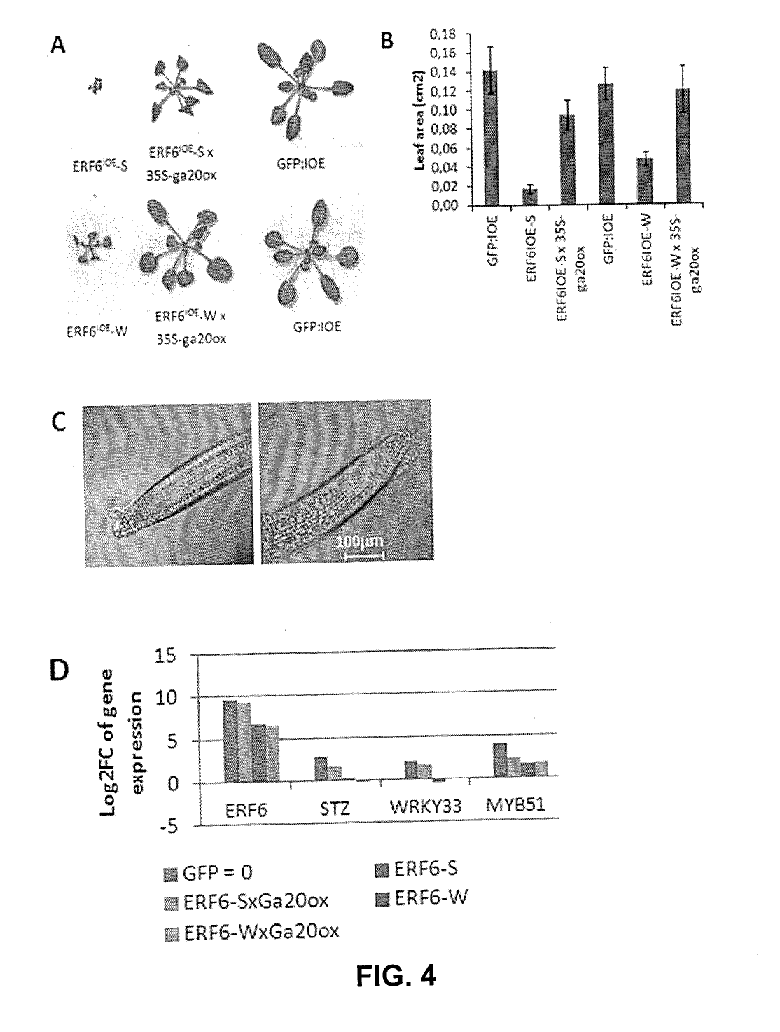

To further explain the cause of reduced leaf size measured upon ERF6 activation, how ERF6 inhibits cell division and expansion was focused upon. Among the putative ERF6 targets, the GA2ox6 gene that encodes for an enzyme responsible for gibberellins degradation was found. It was thus hypothesized that ERF6 overexpression causes a decrease in GA level, which could in turn lead to stabilization of DELLAs and as consequence inhibit cell expansion and division. To test this hypothesis, both ERF6:IOE lines were crossed with a transgenic line overexpressing the GA biosynthetic enzyme GA20-oxidase 1 (GA20-ox1. For ERF6^{IOE}-S, this resulted in partial complementation of the severe growth phenotype, while for ERF6^{IOE}-W, this complementation was total (FIG. 4, Panels A and B). Detailed cellular analysis revealed that this growth complementation resulted from more and larger cells in transgenic line overexpression for both EFR6 and GA20-ox1. The cell area was no longer affected by ERF6 when Ga20-ox is ectopically expressed and the number of cells almost reached the wild-type numbers again.

To further demonstrate the stabilization of the DELLAs upon ERF6 overexpression, the ERF6:IOE lines were crossed with a pRGA-GFP:RGA marker line. Again, the obtained seeds were allowed to grow on control medium until 9DAS and then seedlings were transferred to DEX to induce ERF6 overexpression. At several time points upon transfer, samples were taken for protein extraction and RGA (a DELLA) levels were measured with Western analysis. This demonstrated the RGA stabilization by ERF6, observed after transfer to DEX. Moreover, the stabilization of RGA in the third leaf could be proven in planta with confocal microscopy at subsequent time points upon transfer to DEX (FIG. 4, Panel C). Together, this data proves that ERF6 stimulates GA breakdown, resulting in stabilization of the DELLA protein, RGA.

Finally, to determine whether the activation of the osmotic stress transcriptional network (with STZ, WRKY33 and MYB51) activated by ERF6 could be uncoupled from the growth response pathway (with GA/RGA), the activity of the stress network was measured in the ERF6-IOE×35S-Ga20-ox1. Interestingly, although the growth of these lines was not affected anymore by ERF6 overexpression, the stress-signaling cascade was still induced, as the transcription level of STZ, WRKY33 and MYB51 were almost as high as in the single ERF6 overexpression line.

Because the stress transcriptional network was almost not affected by the ectopic overexpression of Ga20-ox1, it can be concluded that the transcriptional network activated by osmotic stress works completely independent of the growth response to stress.

### 5. ERF6 and Stress Tolerance

As ERF6 was demonstrated to play a central role in signaling and growth regulation upon osmotic stress, growth under stress of ERF6 loss- and gain-of-function lines was investigated. It was opted to study growing plants upon exposure to mild stress, not threatening plant survival but only reducing plant growth by an average of 50%. For loss-of-function analysis, the double erf5/erf6 T-DNA insertion mutant and a wild-type line were grown on control medium until 9DAS and then transferred to growth medium containing 25 mM of the osmoticum Mannitol. Subsequently, upon transfer, growth of the third leaf was measured. While wild-type plants showed the expected growth reduction caused by mild osmotic stress, the growth of erf5/erf6 mutants was less affected. Consistently with the observations from the ERF6 loss-of-function lines, the ERF610E-W line was shown to be hypersensitive to short-term mild osmotic stress.

### 6. Expression of a Gibberellin Biosynthesis Gene Under the ERF6 Promoter in Arabidopsis Thaliana

A chimeric gene is constructed containing the following DNA elements:
ERF6 promoter from Arabidopsis thaliana (depicted in SEQ ID NO:1)
A sense RNA encoding GA 20 oxidase 1 from Arabidopsis thaliana (depicted in SEQ ID NO:2)
A CaMV 35S terminator

This chimeric gene is introduced into a T-DNA vector (pK7 m24GW-FAST), together with a selectable GFP marker. The T-DNA vector is introduced into Agrobacterium tumefaciens and used to produce transgenic Arabidopsis.

Leaf growth of the transgenic is plants is analyzed under optimal and stress conditions.

Wild-type and transgenic seeds are grown in vitro with Murashige and Skoog (MS) medium containing 0.5% sucrose under a 16-hour/8-hour photoperiod. For osmotic stress, wild-type and transgenic seeds are allowed to germinate for 5 to 7 days and transferred to mannitol containing agar plates (Skirycz et al. 2010). Shoot fresh and dry weight, leaf area, root length and mass are measured. Under soil conditions, a high-throughput, fully automated water monitoring system, named WIWAM, implemented at the host institute is used (Skirycz et al. 2011). This system enables keeping water levels stable and is capable of taking digital images of individual plants that can be used to determine rosette growth, leaf area and leaf shape. Plants are grown under controlled watering regimen until stage 1.04 (approximately 12-13 days old), after which control or limited watering are applied for additional 10-12 days. At the end of the experiment, plants are harvested and the shoot production is recorded as a measurement of yield.

### 7. Modulation of the Gibberellin Production in Corn

A chimeric gene is constructed containing the following DNA elements:
ERF6 promoter from Zea mays (depicted in SEQ ID NO:3)
a sense GA 20 oxidase I from Zea mays (depicted in SEQ ID NO:4)
a CaMV 35S terminator

The chimeric gene is introduced into the destination vectors (pBbm42GW7), containing the BASTA herbicide under control of 35S CaMV promoter and followed by a nos terminator as a selectable marker. The constructs are introduced into Agrobacterium tumefaciens (EHA 101) and used to transform immature maize embryos of B 104, which are regenerated by tissue culture to produce transgenic plants (i.e., transgenic maize plants expressing the GA 20 oxidase 1 when abiotic stress is perceived by Zea mays). The transgenic plants are backcrossed to B104, resulting in a working population segregating in 50% sensitive and thus control plants and 50% transgenic plants. Leaf growth of the segregating population is analyzed under optimal and drought stress conditions. The plants are grown in soil and watered daily: the drought-treated plants receive 70% of the water that is added to the control plants. The leaf growth is monitored by daily measuring the leaf length of the fourth leaf upon its appearance, providing data on the leaf elongation rate and the final leaf length. In addition, final plant height, fresh weight and dry weight plants will be determined as a measure for plant biomass.

### Materials and Methods

### 1. Plant Lines

The inducible ERF6 overexpression lines described here were kindly provided by Dr. Youichi (RIKEN—Japan). The pRGA:RGA-GFP line was a kind gift of Prof. Dr. Tai-ping Sun (Duke University, Durham, N.C., USA). All lines used are in Col-0 background.

### 2. Plant Growth Conditions

Seedlings were grown in vitro on half-strength Murashige and Skoog medium (Murashige and Skoog 1962) containing 1% sucrose and 6.5 g/L agar at 21° C. under a 16-hour day (110 μmol m⁻² s⁻¹) and 8-hour night regimen. The growth medium was overlaid with nylon mesh (Prosep, Zaventem, Belgium) of 20 μm pore size to facilitate transfer to induction medium. For expression analysis and growth experiments, 64 resp. 32 seeds were equally distributed on a 15-cm diameter petri dish. Strong and weak ERF6-overexpressing plants as well as controls were always grown together on one plate to enable correct comparisons.

### 3. Induction of ERF6 Overexpression

At 9DAS, when the third leaf is fully proliferating, the mesh with seedlings was transferred to plates containing ½ MS medium with 5 μM Dexamethasone (Dex). For expression analysis and growth experiments, all seedlings were transferred to Dex, including the control lines, to account for the possible effects of Dex on growth or gene expression. Because no suitable control line was available for the experiments with crosses, and as Dex was shown in these experiments to have no effect on growth or gene expression, half of the seedlings were transferred to Dex and the other half was transferred to ½ MS medium without Dex. Here, the non-treated plants will serve as a control for the Dex-induced plants of the same line. For the growth analysis of ERF6:GR×35S:GA200×1, plants were grown without mesh on ½ MS with Dexamethasone from the beginning.

### 4. Growth Analysis

Growth analysis was performed on the third true leaf harvested at different time points after transfer to Dex. After clearing with 70% ethanol, leaves were mounted in lactic acid on microscopic slides. For each experiment, about 15-20 leaves were photographed with a binocular, and abaxial epidermal cells (100-200) were drawn for three representative leaves with a DMLB microscope (Leica) fitted with a drawing tubus and a differential interference contrast objective. Photographs of leaves and drawings were used to measure leaf area and cell size, respectively, using ImageJ v1.37o (NIH; on the World Wide Web at //rsb.info.nih.gov/ij/), and from these cell numbers were calculated by dividing leaf area with cell area.

### 5. Sampling RNA for Expression Analysis

Leaf 3 was harvested from plants at 1, 2, 4 and 24 hours after transfer. Samples were obtained from three independent experiments and from multiple plates within the experiment. Whole seedlings were harvested rapidly in an excess of RNAlater solution (Ambion) and, after overnight storage at 4° C., dissected under a binocular microscope on a cooling plate with precision microscissors. Dissected leaves were transferred to a new tube, frozen in liquid nitrogen, and ground with a Retsch machine and 3-mm metal balls. RNA was extracted with TriZol (Invitrogen) and further purified with the RNEASY® Mini Kit (Qiagen). DNA digestion was done on-column with Rnase-free DNase I (Roche).

### 6. Genome-Wide Expression Changes

For genome-wide expression changes, samples of the strong ERF6 overexpressing line and the control line harvested 4 hours after transfer to Dex were used. Two μg of pure RNA samples were hybridized to AGRONOMICS 1 Arabidopsis Tiling Arrays at the VIB Microarray Facility (Leuven, Belgium). Obtained expression data was processed with Robust Multichip Average (RMA) (background correction, normalization, and summarization) as implemented in BioConductor (Irizarry et al. 2003a; Irizarry et al. 2003b; Gentleman et al. 2004). As cdf, “agronomicslattairtcdfl” was used, in which several probes belonging to 1 gene are pooled to calculate one expression value per gene. The BioConductor package Limma was used to identify differentially expressed genes (Smyth 2004). A factorial design (ERF6:GR-GFP:GR) was applied to analyze the data. For comparisons of interest, moderated t statistics were calculated using the eBayes function and P values were corrected for multiple testing for each contrast separately using topTable (Hochberg and Benjamini 1990). FDR-corrected p-value<0.05 was used as a cut-off.

### 7. Flow Cytometry

For flow cytometry analysis, 4-32 leaves were chopped with a razor blade in CyStain UV Precise P buffers (Partec) according to the manufacturer's instructions. The nuclei were analyzed with a CyFlow flow cytometer with the FloMax Software (Partec, Miinster, Germany).

8. qRT-PCR For cDNA synthesis, the iScript cDNA Synthesis Kit (Biorad) was used according to the manufacturer's instructions using 1 μg of RNA. Primers were designed with the QuantPrime website (Arvidsson et al. 2008). qRT-PCR was done on a LIGHTCYCLER® 480 (Roche Diagnostics) in 384-well plates with LIGHTCYCLER® 80 SYBR Green I Master (Roche) according to the manufacturer's instructions. Melting curves were analyzed to check primer specificity. Normalization was done against the average of housekeeping genes GAPDH and CBP20; ΔCt=Ct (gene)−Ct (mean (housekeeping genes)) and ΔΔCt=ΔCt(GFP:GR)−ΔCt(ERF6:GR). Ct refers to the number of cycles at which SYBR Green fluorescence reaches an arbitrary value during the exponential phase of the cDNA amplification.

### 9. GUS Staining

For GUS-experiments, 14-day-old seedlings treated and non-treated with Dex for 72 hours were harvested in heptane and incubated for 10 minutes. Seedlings were washed in 100 mM Tris-HCl/50 mM NaCl (pH 7.0), and subsequently incubated in 5-bromo-4-chloro-3-indolyl-3-D-glucuronide (X-Gluc) buffer [100 mM Tris-HCl/50 mM NaCl buffer (pH 7.0), 2 mM K₃-[Fe(CN)₆] and 4 mM X-Gluc]) at 37° C. for 24 hours. Seedlings were washed in 100 mM Tris-HCl/50 mM NaCl (pH 7.0) and bleached in subsequently 50% and 100% ethanol followed by mounting in lactic acid. Samples were photographed with a differential interference contrast microscope (Leica, Vienna, Austria).

### 10. RGA:GFP Quantification

Amounts of RGA:GFP protein in either Dex-treated or non-treated ERF6:GR plants were quantified by Western Blotting. Complete seedlings were harvested in liquid nitrogen 48 hours upon transfer to Dex or control medium and ground with the Retsch Machine. Protein extraction was done by adding extraction buffer (Van Leene et al. 2007) to ground samples, followed by two freeze-thaw steps and two centrifugation steps (20,817 g, 10 minutes, 4° C.) whereby the supernatant was collected each time. Western blot analysis was performed with primary rabbit anti-GFP antibodies (Santa Cruz, Calif., USA) (diluted 1:200) and a secondary horseradish peroxidase-conjugated donkey anti-rabbit antibodies (GE-Healthcare) (diluted 1:10000). A chemiluminescence procedure (NEN Life Science Products) was used for detection.

1. A method for producing an abiotic stress tolerant plant relative to a control plant, the method comprising:
introducing and expressing a chimeric gene in the plant, wherein the chimeric gene comprises a stress inducible promoter operably linked to a gibberellin biosynthesis gene.

2. A method according to claim 1 wherein the stress inducible promoter is derived from a gene selected from the group consisting of ERF6, TCH3, embryo-abundant protein-related, ankyrin repeat family protein, At1g05575, calcium-binding protein, glycine-rich protein, At1g19020, EDA39, STZ, MYB51, WRKY33, ERF1, ERF2, ERF5, ERF11, and an orthologous gene of any thereof.

3. A method according to claim 1 wherein the giberrellin biosynthesis gene is selected from the group consisting of GA 20 oxidase 1 (GA 20ox1), GA 3 oxidase1 (GA 3ox1) and ent kaurenoic acid oxidase (KAO).

4. A chimeric gene comprising the following operably linked DNA elements:
a) a stress inducible promoter,
b) a DNA region encoding for a giberrellin biosynthesis gene, and
c) a 3′ end region comprising transcription termination and polyadenylation signals functional in a plant cell.

5. The chimeric gene of claim 4, wherein the stress inducible promoter is derived from a gene selected from the up consisting of ERF6, TCH3, embryo-abundant protein-related, ankyrin repeat family protein, At1g05575, calcium-binding protein, glycine-rich protein, At1g19020, EDA39, STZ, MYB51, WRKY33, ERF1, ERF2, ERF5, ERF11, and an orthologous gene of any thereof.

6. The chimeric gene of claim 4, wherein the giberrellin biosynthesis gene is selected from the group consisting of GA 20 oxidase 1 (GA20ox1), GA 3 oxidase1 (GA 3ox1) and ent kaurenoic acid oxidase (KAO).

7. A transgenic plant, a transgenic seed, or a transgenic plant cell comprising the chimeric gene of claim 4.

8. The transgenic plant, transgenic seed, or transgenic plant cell of claim 7, wherein the plant, seed or cell is selected from the group consisting of a crop plant, a monocot, a cereal, rice, wheat, maize, barley, millet, rye, triticale, sorghum emmer, spelt, secale, einkorn, teff, milo and oat.

9. A transgenic plant, a transgenic seed, or a transgenic plant cell comprising the chimeric gene of claim 5.

10. The transgenic plant, transgenic seed, or transgenic plant cell of claim 9, wherein the plant, seed or cell is selected from the group consisting of a crop plant, a monocot, a cereal, rice, wheat, maize, barley, millet, rye, triticale, sorghum emmer, spelt, secale, einkorn, teff, milo, and oat.

11. A transgenic plant, a transgenic seed, or a transgenic plant cell comprising the chimeric gene of claim 6.

12. The transgenic plant, transgenic seed, or transgenic plant cell of claim 11, wherein the plant, seed or cell is selected from the group consisting of a crop plant, a monocot, a cereal, rice, wheat, maize, barley, millet, rye, triticale, sorghum emmer, spelt, secale, einkorn, teff, milo, and oat.
